(19) United States
(12) Patent Application Publication
(10) Pub. No.: US 2007/0238096 A1
(43) Pub. Date: Oct. 11, 2007

(54) Hybrid energy transfer for nucleic acid detection

(75) Inventors: Norbert O. Reich, Santa Barbara, CA (US); R. August Estabrook, Goleta, CA (US); Gary Braun, Santa Barbara, CA (US)

Correspondence Address: HELLER EHRMAN LLP, 275 MIDDLEFIELD ROAD, MENLO PARK, CA, 94025-3506, US

(73) Assignee: The Regents of the University of California

(21) Appl. No.: 11/716,491

(22) Filed: Mar. 9, 2007

Related U.S. Application Data
(60) Provisional application No. 60/780,997, filed on Mar. 9, 2006.

(51) Int. Cl. C12Q 1/70; C12Q 1/68; C12P 19/34; C07H 21/02

(52) U.S. Cl. 435/5; 435/6; 435/91.2; 536/25.32

(57) ABSTRACT

Assays using enzymatic degradation of RNA/DNA heteroduplexes are provided for the detection of target nucleic acid molecules. Such enzymatic degradation may be obtained by enzymes including RNaseH. Exemplary methods include Probe Trapping (PT), Hybrid Energy Transfer (HET), and Fluorescent Probe Degradation (FPD). The assays make use of an RNA or chimeric RNA/DNA probe which recognizes a target DNA sequence and forms an RNA/DNA heteroduplex that is the substrate for the enzyme RNaseH which degrades the RNA portion of the probe. Degraded probe fragments diffuse away from the DNA target leading to a detectable signal and allowing the DNA target to hybridize to another probe. Probe degradation is cycled over time. The assays disclosed herein are suitable for use in detecting DNA sequences and could be used in a medical diagnostic.

### CROSS-REFERENCE TO RELATED APPLICATIONS

This application claims the benefit of priority under 35 U.S.C. § 119(e) from Provisional Application 60/780,997 filed Mar. 9, 2006, the entire contents of which are hereby incorporated by reference in their entirety.

### FIELD OF THE INVENTION

The inventions disclosed herein provide methods and compositions for detecting and identifying target nucleic acid molecules such as deoxyribonucleic acid (DNA) or ribonucleic acid (RNA). Methodology is also described for sequence-specific nucleic acid identification.

### BACKGROUND

The need for efficient assays which detect specific DNA sequences is enormous and constantly growing. The recent increases in the number of sequenced genomes for bacterial and viral pathogens as well as humans provide the potential for improved medicine and diagnostics. For example, molecular beacon-based nucleic acid detection platforms are actively being pursued in basic research, and are now emerging as the basis of powerful new sensing and diagnostic methods (J. Z. Jordens, S. Lanham, M. A. Pickett, S. Amarasekara, I. Abeywickrema, P. J. Watt, Amplification with Molecular Beacon Primers and Reverse Line Blotting for the Detection and Typing of Human Papillomaviruses. Journal of Virological Methods 89, 29-37, (2000)). However, optical detection requires high beacon concentrations and low clinical levels of target DNA are frequently problematic for such systems. Usually signal amplification (such as enzyme-linked immunosorbent assay (ELISA) methods) or target amplification (such as by polymerase chain reaction (PCR)) is required, both of which have drawbacks. The disclosed inventions provide a means for identifying and quantization of the amount of a given nucleic acid sequence in a given sample. Furthermore, these assays are sequence-specific and only give signal when the correct target sequence is present. Three assays are described with various embodiments.

### SUMMARY

Disclosed herein are assays for detection of target molecules, such as target nucleic acids, using enzymes that recognize RNA/DNA hetero-duplexes. The assays provided by the disclosure may be used in many embodiments to detect sequence-specific nucleic acids. Disclosed herein are different embodiments of assays using enzymatic degradation of RNA/DNA heteroduplexes; such enzymatic degradation may be obtained by using RNaseH (also known as, e.g., ribonuclease H and endoribonuclease H) or other enzymes including, but not limited to, RNaseI, RNaseIII, Nuclease S1, T7 Exonuclease, and Exonuclease III (Exo III), or any combination thereof. Exemplary methods include Probe Trapping (PT), Hybrid Energy Transfer (HET), and Fluorescent Probe Degradation (FPD). All of these exemplary technologies make use of an RNA or chimeric RNA/DNA probe which recognizes a target nucleic acid sequence and forms an RNA/DNA heteroduplex. This heteroduplex comprises the substrate for the enzymatic cleavage of the probe leading to a detectable signal.

The invention provides a method for detecting a single-stranded or double-stranded target nucleic acid. The method includes (a) contacting a sample comprising a target nucleic acid with an oligonucleotide probe preparation to form a reaction mixture under conditions that allow an oligonucleotide probe in the oligonucleotide probe preparation to hybridize to the target nucleic acid to form a probe-target complex, wherein the oligonucleotide probe preparation comprising a plurality of oligonucleotide probes and an agent that selectively cleaves the oligonucleotide probes upon forming a complex with the target nucleic acid, wherein the oligonucleotide probe comprises one or more analog fluorescence bases; (b) maintaining the reaction mixture for a sufficient amount of time to allow reaction of the reaction mixture with the target nucleic acid; and (c) detecting fluorescence in the sample, wherein fluorescence is indicative of the presence of the target nucleic acid. In one embodiment, the analog fluorescence base is selected from the group consisting of 2-aminopurine (AP), pyrrolo-dC (P-dC), 6-Methyl-3-(β-D-2-deoxyribofuranosyl)pyrrolo[2,3-d]pyrimidin-2-one (pyrrolo cytosine), 4-amino-7-oxo pteridine, 4-amino-6-methyl-7-oxo pteridine, 2-amino-4,7-oxo pteridine, 2,4-oxo pteridine, 2-dimethyl amino-7-oxo pteridine, 3-methyl-isoxanthopterin, and any combination thereof. In another embodiment, the method is performed in a microfluidics device. The target nucleic acid can be any oligonucleotide or polynucleotide of any organism (e.g., the target nucleic acid comprises a genome or fragment of the genome from Human Papillomavirus (HPV)). In one embodiment, the oligonucleotide probe comprises a sequence of about 15 to 30 nucleotides and contains the deoxynucleotide sequence CTAAAACGAAAGTA (SEQ ID NO: 1) or the complement thereof TACTTTCGTTTTAG (SEQ ID NO: 2), or a ribonucleotide sequence CUAAAACGAAAGUA (SEQ ID NO: 3) or the complement thereof UACUUUCGUUUUAG (SEQ ID NO: 4), or any mixture of the two wherein one or more bases of the oligonucleotide probe comprise a fluorescent analog base.

The invention also provides a method for detecting a single-stranded or double-stranded target nucleic acid. The method includes (a) contacting a sample comprising a target nucleic acid with an oligonucleotide probe preparation to form a reaction mixture under conditions that allow an oligonucleotide probe in the oligonucleotide probe preparation to hybridize to the target nucleic acid to form a probe-target complex, wherein the oligonucleotide probe preparation comprising a plurality of oligonucleotide probes and an agent that selectively cleaves the probes upon forming a complex with the target nucleic acid, wherein the oligonucleotide probe generates a detectable signal change upon cleavage with the agent; (b) maintaining the reaction mixture for a sufficient amount of time to allow reaction of the reaction mixture with the target nucleic acid; and (c) detecting a detectable signal in or emanating from the sample, wherein the detectable signal is indicative of the presence of the target nucleic acid. For example, in one aspect, the plurality of oligonucleotide probes comprise the general structure: X-NA₁-R-NA₂-Y, or X-NA₁-R, X-NA₁-R-NA₂, or X-R-NA₂ wherein NA₁ and NA₂ comprise polyethylene glycol (PEG) (or other non-proteinaceous polymer) linkers, DNA, RNA, peptide nucleic acid (PNA), locked nucleic acid (LNA) nucleotides or a combination thereof having a length of about 3-100 nucleotides in length, wherein R is a scissile nucleic acid linkage or RNA of about 1-100 ribonucleotides in length, wherein either or both of X and Y generate a detectable signal, wherein X and Y when linked by NA₁-R-NA₂ (i) do not generate a detectable signal, or (ii) generate a signal indicative of an uncleaved probe, whereby upon cleavage with the agent at least one intact oligonucleotide fragment or degraded probe fragment is generated, such fragment being, or being treated so as to be, no longer capable of remaining hybridized to the target nucleic acid, wherein either or both of X and Y generate a detectable signal.

The invention further provides a method for detecting a single-stranded or double-stranded target nucleic acid which comprises: (a) contacting a sample comprising a target nucleic acid with an oligonucleotide probe preparation under conditions that allow an oligonucleotide probe in the oligonucleotide probe preparation to hybridize to the target nucleic acid to form a probe-target complex; (b) contacting the sample comprising the probe preparation with an agent to form a reaction mixture, wherein the agent selectively cleaves oligonucleotide probes upon forming the probe-target complex; (c) maintaining the reaction mixture for a sufficient amount of time to allow reaction of the oligonucleotide probes with the target nucleic acid and the agent; (d) neutralizing the agent; (e) contacting the reaction mixture with a trapping agent comprising at least one complementary oligonucleotide linked to a substrate, wherein the complementary oligonucleotide is complementary to at least one intact oligonucleotide probe, under conditions wherein at least one complementary oligonucleotide hybridizes to at least one intact oligonucleotide probe to form a trapping agent-probe complex; (f) separating the trapping agent-probe complex from the reaction mixture; and (g) detecting a detectable signal in or emanating from the reaction mixture, wherein the presence of a detectable signal is indicative of the presence of the target nucleic acid, wherein the oligonucleotide probe comprises a detectable moiety.

The invention provides a method for detecting a single-stranded or double-stranded target nucleic acid. The method comprising (a) contacting a sample comprising a target nucleic acid with an oligonucleotide probe preparation under conditions that allow an oligonucleotide probe in the oligonucleotide probe preparation to hybridize to the target nucleic acid to form a probe-target complex; (b) contacting the sample comprising the probe preparation with an agent to form a reaction mixture, wherein the agent selectively cleaves oligonucleotide probes upon forming the probe-target complex; (c) maintaining the reaction mixture for a sufficient amount of time to allow reaction of the oligonucleotide probes with the target nucleic acid and the agent; (d) contacting the reaction mixture with a trapping agent comprising a complementary oligonucleotide that hybridizes to intact oligonucleotide probes to form a trapping agent-probe complex, wherein the trapping-agent probe complex does not react with the agent; (e) separating the trapping agent-probe complex from the reaction mixture; and (f) detecting a detectable signal in or emanating from the reaction mixture, wherein the presence of a detectable signal is indicative of the presence of the target nucleic acid, wherein the oligonucleotide probe comprises a detectable moiety.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

FIG. 1 is a schematic illustration of an embodiment of the Probe Trapping (PT) assay showing a probe having a fluorescein dye at one end and a biotin molecule at the other end, the probe capable of hybridizing with a target DNA molecule (double-stranded in this example), and leading to the generation of a fluorescence signal following enzymatic degradation by RNaseH of an RNA probe having a dye and a biotin moiety used to remove intact probes. D indicates dye, and B indicates biotin. Probe degradation is indicated by the term RNA dNTP's indicating free dNTP's (deoxyribonucleotide triphosphates) released by degradation of the RNA of the probe.

FIG. 2 illustrates a further exemplary scheme for Hybrid Energy Transfer (HET) assays. D indicates a fluorescent dye such as, for example, fluorescein. NP indicates a nanoparticle that is suitable for quenching fluorescence from the dye, such as a metallic (e.g., gold) nanoparticle. Probe degradation is indicated by the term “RNA dNTP's” indicating free dNTP's (deoxyribonucleotide triphosphates) released by degradation of the RNA of the probe.

FIG. 3 is a schematic illustration of an embodiment of an assay having features of the invention utilizing Fluorescent Probe Degradation. In this formulation, fluorescent base analogs are incorporated into the probe. These base analogs are heavily quenched when placed into a nucleic acid polymer and emit well as free nucleotides, not stacked in a nucleic acid polymer. Probe degradation is indicated by the term “RNA dNTP's” indicating free dNTP's (deoxyribonucleotide triphosphates) released by degradation of the RNA of the probe. Thus, degraded probes give rise to an increase in fluorescent signal.

FIG. 4 is a schematic illustration of an embodiment of an assay having features of the invention utilizing Hybrid Energy Transfer detecting a DNA target from a lysed cell. As indicated in this schematic illustration, cells are lysed and the DNA target extracted followed by probe and enzyme addition. Degraded probe accumulates linearly over time as fluorescent signal.

FIG. 5 shows a polyacrylamide gel electrophoresis (PAGE) assay with five lanes confirming that probes are intact before the assay (lane 1), can hybridize to DNA targets (lane 2 to 3), and are degraded by RNaseH (lane 4 and 5).

FIG. 6 is a Forster plot calculation for fluorescein illustrating quenching by both 6 nm and 13 nm gold nanoparticles illustrating the effect of gold nanoparticle size on quenching efficiency for fluorescein. This data shows the larger distances attainable with a dye/nanoparticle system as opposed to traditional FRET dye pairs. This greater distance dependence with efficient energy transfer makes it possible for use with sequences specific nucleic acid probes (5-100 base pairs). Energy transfer between dyes and nanoparticles does not follow traditional FRET mechanisms (distance dependence of 1/r⁶), has been reported to be SET mechanisms (distance dependence of 1/r⁴), and these two mechanisms of energy transfer are fundamentally distinct (Nanometal Surface Energy Transfer in Optical Rulers, Breaking the FRET Barrier, C. S. Yun, A. Javier, T. Jennings, M. Fisher, S. Hira, S. Peterson, B. Hopkins, N, O. Reich, and G. F. Strouse, J. Am. Chem. Soc. 127(9), pp. 3115-9, (2005)).

FIG. 7 shows fluorescence data obtained from PT experiments performed according to the scheme illustrated in FIG. 1. 1 pmol PT probe was incubated with varying amounts of DNA and 0.5 units thermostable RNaseH for 2 hours at 61° C. The small dashed line is buffer (0 pM), dotted line is 100 nM, dashed line (large dashes) is 10 nM, dashed-dotted line is 1 nM, and the solid line is 100 pM. Inset shows the area of each plot. Data was repeated, averaged, and summarized in FIG. 8.

FIG. 8 illustrates the current sensitivity of an embodiment of the PT assay with synthetic DNA targets shown in FIG. 7. Concentrations of target, measured signal, and signal-to-noise calculations are reported. 1 pmol PT probe was incubated with varying amounts of DNA and 0.5 units thermostable RNaseH for 2 hours at 61° C.

FIG. 9 shows spectra from an embodiment of the HET assay. 10 fmol (1 nM) of HET probe was incubated with varying amounts of synthetic DNA with 0.043 units of E. coli RNaseH for 1 hour incubation at 37° C. This data was replicated, averaged, and summarized in FIG. 10.

FIG. 10 is a summary of fluorescence data from an embodiment of the HET assay. * is with no enzyme. 10 fmol (1 nM) of HET probe was incubated with varying amounts of synthetic DNA with 0.043 units of E. coli RNaseH for 1 hour incubation at 37° C.

FIG. 11 is data from an embodiment of the PT assay using DNA plasmid. Only DNA with the target sequence triggers probe degradation and consequent signal production.

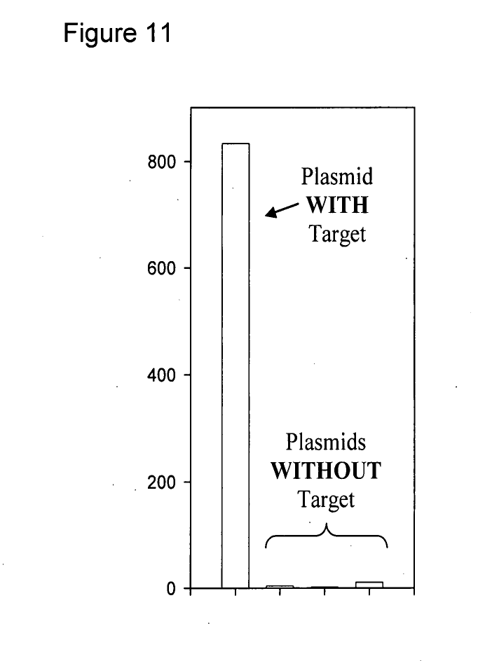

FIG. 12 presents data from an embodiment of the HET assay using DNA plasmid as the target. 0.9 nM HET probe was mixed with 1 μL of E. coli RNaseH from Promega and incubated for 1 hour at 37° C. Intensity and signal-to-background (S/B) are reported.

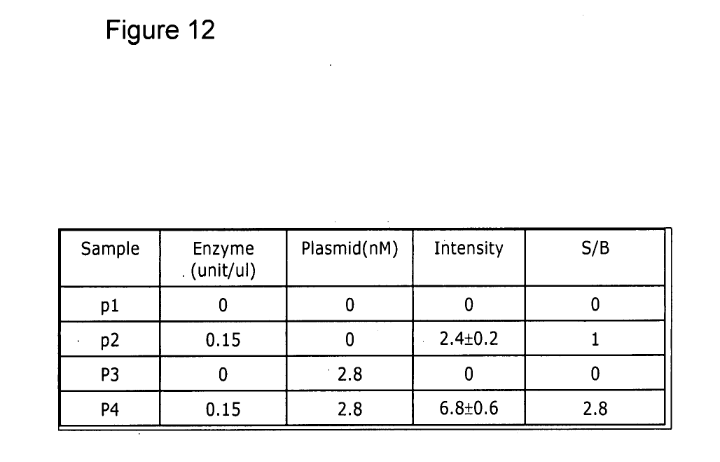

FIG. 13 schematically illustrates an exemplary layout of a microfluidic chip suitable for use with the methods disclosed herein. The sample and reagents are directed through to the mixing region. Magnets can be used to separate reagents and components when needed. The sample is measured at the fluorescence detection region and then directed to the waste.

FIG. 14 schematically illustrates an example of a standard fabrication process for microfluidic chips. The microfluidic channels are formed using standard soft lithography PDMS processes. Nickel micro strips are patterned using e-beam lithography and a lift-off process.

### DETAILED DESCRIPTION

As used herein and in the appended claims, the singular forms “a,” “and,” and “the” include plural referents unless the context clearly dictates otherwise. Thus, for example, reference to “an oligonucleotide” includes a plurality of such oligonucleotides and reference to “the enzyme” includes reference to one or more enzymes known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Disclosed herein are assays for detection of target molecules, such as target nucleic acids, using enzymes that recognize RNA/DNA hetero-duplexes. The assays provided by the disclosure may be used in many embodiments to detect sequence-specific nucleic acids. Disclosed herein are different embodiments of assays using enzymatic degradation of RNA/DNA heteroduplexes; such enzymatic degradation may be obtained by using RNaseH or other enzymes including, but not limited to, RNaseI, RNaseIII, Nuclease S1, T7 Exonuclease, and Exonuclease III (Exo III), or any combination there of. Exemplary methods include Probe Trapping (PT), Hybrid Energy Transfer (HET), and Fluorescent Probe Degradation (FPD). All of these exemplary technologies make use of an RNA or chimeric RNA/DNA probe which recognizes target polynucleotides and forms an RNA/DNA heteroduplex. This heteroduplex comprises the substrate for the enzymatic cleavage of the probe leading to a detectable signal.

The inventions provide methods and systems for identifying and quantization of the amount of a given nucleic acid sequence in a given sample. Furthermore, the methods and systems of the invention provides are sequence specific and provide a detectable signal when the correct target sequence is present. The disclosure provides various embodiments of the invention.

Useful background for understanding and practicing the present invention may be found in U.S. Pat. No. 6,811,973, No. 5,403,711, No. 6,875,572, No. 4,983,728, No. 4,876,187, No. 6,706,471, No. 7,011,944, and No. 5,011,769. Other published work that may help to understand the present disclosure includes: 1) Nanometal Surface Energy Transfer in Optical Rulers, Breaking the FRET Barrier, C. S. Yun, A. Javier, T. Jennings, M. Fisher, S. Hira, S. Peterson, B. Hopkins, N, O. Reich, and G. F. Strouse, J. Am. Chem. Soc. 127(9), pp. 3115-9, (2005); 2) Spermine-mediated improvement of cycling probe reaction, Z. Modrusan, F. Bekkaoui, P. Duck, Mol. Cell Probes, 12, 107-116, (1998); 3) Direct Detection of Genomic DNA by Enzymatically Amplified SPR Imaging Measurements of RNA Microarrays, T. T. Goodrich, H. J. Lee, and R. M. Corn, J. Am. Chem. Soc., 126, 4086-4087, (2004); 4) Self-Assembled Nanoparticle Probes for Recognition and Detection of Biomolecules, D. J. Maxwell, J. R. Taylor, and S. Nie, J. Am. Chem. Soc., 124, 9606-9612, (2002); 5) Gold Nanoparticle Based FRET Assay for the Detection of DNA Cleavage, P. C. Ray, A. Fortner, and G. K. Darbha, J. Phys. Chem. B, 110, 20745-20748 (2006), 6) Genomic DNA Detection Using Cycling Probe Technology and Capillary Gel Electrophoresis with Laser-Induced Fluorescence, T. D. Lang, D. C. W. Mah, R. T. Poirier, F. Bekkaoui, W. E. Lee, D. E. Bader, Molecular and Cellular Probes, 18, 341-348, (2004). 7) Probe Amplifier System Base on Chimeric Cycling Oligonucleotides, P. Duck, G. Alvarado-Urbina, B. Burdick, and B. Collier, Biotechniques, 9, 142-146, (1990). 8) Identification of RNase HII from psychrotrophic bacterium, Shewanella sp. SIB1 as a high-activity type RNase H., H. Chon, T. Tadokoro, N. Ohtani, Y. Koga, K. Takano, and S. Kanaya, FEBS J., 10, 2264-75 (2006). 9) Kinetic characteristics of Escherichia coli RNase Hi: cleavage of various antisense oligonucleotide RNA duplexes, S. T. Crooke, K. M. Lemonidis, L. Neilson, R. Griffey, E. A. Lesnik, and B. P. Monia, J. Biochem. 312, 599-608, (1995). All patents and publications cited, both supra and infra, are hereby incorporated by reference in their entireties.

Probes useful for biomolecular target identification (e.g. Molecular Beacons or MBs) comprise a nucleic acid identification component and a signaling component. A nucleic acid identification component comprises oligonucleotides or polynucleotides substantially complementary to a target polynucleotide. An oligonucleotide typically comprises a polymer of nucleic acids from 10-500 (e.g. 10-20, 20-30, 30-40, 40-50, 50-100, 100-150, 150-300, or 300-500 nucleic acids in length). Such nucleic acids can comprise DNA or RNA or nucleic acid base analogs. Nucleic acid components are nearly identical for all assays utilizing sequence-specific probes and are predominantly composed of DNA, RNA, LNA, or PNA. With 5′, 3′, and internal modifications, many options for signaling are possible. Signaling components of probes can be through a radiochemical moiety (examples: ³²P, ³⁵S, ³H), affinity moiety (examples: Biotin, Digoxygenin, Thiol), electrical moiety (example: methylene blue with gold electrode surface), or an optical moiety (examples: Fluorescein, Cy3, Alexa488, Quantum Dots) Optical moieties are usually organic and synthetic fluorophores or quantum dots (QDs). QDs enable long-range, high intensity, multicolor labeling of cellular molecules or probes. MBs provide a powerful and specific synthetic probe strategy with single base-mismatch discrimination.

A signaling component can include any label that can be detected optically, electronically, radioactively and the like. A nucleic acid analog may serve as the signaling component. By “label” or “detectable label” is meant a moiety that allows detection. In one embodiment, the detection label is a primary label. A primary label is one that can be directly detected, such as a fluorophore. In general, labels fall into three classes: a) isotopic labels, which may be radioactive or heavy isotopes; b) magnetic, electrical, thermal labels; and c) colored or luminescent dyes. Common labels include chromophores or phosphors but are typically fluorescent dyes. Suitable dyes for use in the disclosure include, but are not limited to; fluorescent lanthamide complexes, including those of Europium and Terbium, fluorescein, rhodamine, tetramethylrhodamine, eosin, erythrosin, coumarin, methyl-coumarins, quantum dots (also referred to as “nanocrystals”), pyrene, Malacite green, stilbene, Lucifer Yellow, Cascade Blue™, Texas Red, Cy dyes (Cy3, Cy5, and the like), Alexa dyes, phycoerythin, bodipy, and others described in the 6th Edition of the Molecular Probes Handbook by Richard P. Haugland, hereby expressly incorporated by reference.

It will be understood that embodiments of the invention include probes having fluorescent dye molecules, fluorescent compounds, or other fluorescent moieties. A dye molecule may fluoresce, or be induced to fluoresce upon excitation by application of suitable excitation energy (e.g., electromagnetic energy of suitable wavelength), and may also absorb electromagnetic energy (“quench”) emitted by another dye molecule or fluorescent moiety. Any suitable fluorescent dye molecule, compound or moiety may be used in the practice of the invention. For example, suitable fluorescent dyes, compounds, and other fluorescent moieties include fluorescein, 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED) and 2′-chloro-7′-phenyl-1,4-dichloro-6-carboxyfluorescein (VIC), cyanine dyes (e.g., Cy³, Cy⁵, Cy⁹, nitrothiazole blue (NTB)), Cys3, FAM™, tetramethyl-6-carboxyrhodamine (TAMRA), tetrapropano-6-carboxyrhodamine (ROX), dipyrromethene boron fluoride (Bodipy), dichloro-fluorescein, dichloro-rhodamine, fluorescein thiosemicarbazide (FTC), sulforhodamine 101 acid chloride (Texas Red), phycoerythrin, rhodamine, carboxytetramethylrhodamine, 4,6-diamidino-2-phenylindole (DAPI), an indopyras dye, pyrenyloxytrisulfonic acid (Cascade Blue), 514 carboxylic acid (Oregon Green), eosin, erythrosin, pyridyloxazole, benzoxadiazole, aminonapthalene, pyrene, maleimide, a coumarin, 4-fluoro-7-nitrobenofurazan (NBD), 4-amino-N-[3-(vinylsulfonyl)-phenyl]naphthalimide-3,6-disulfonate) (Lucifer Yellow), DABCYL, DABSYL, anthraquinone, malachite green, nitrothiazole, and nitroimidazole compounds, propidium iodide, porphyrins, lanthamide cryptates, lanthamide chelates, derivatives and analogs thereof (e.g., 5-carboxy isomers of fluorescein dyes), and other fluorescent dyes and fluorescent molecules and compounds.

Probes of the disclosure are designed to have at least a portion be substantially complementary to a target polynucleotide, such that hybridization of the target polynucleotide and the probes of the disclosure occur. As outlined below, this complementarity need not be perfect; there may be any number of base pair mismatches which will interfere with hybridization between the target polynucleotide and the single stranded hybridization probe of the disclosure. Thus, by “substantially complementary” herein is meant that the probes are sufficiently complementary to the target polynucleotide to hybridize under moderate to high stringency conditions. Ideally, the probe is 100% complementary to a target polynucleotide over a stretch of about 10-500 nucleotides.

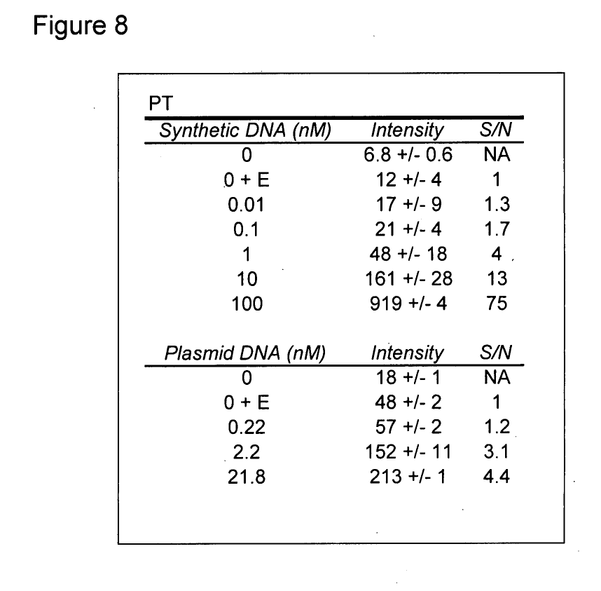

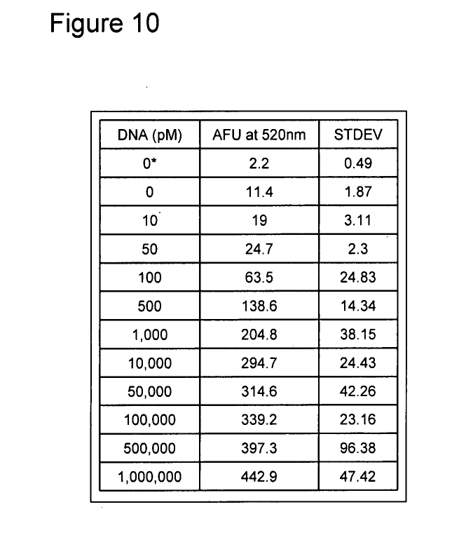

Any enzyme capable of cleaving RNA in a RNA/DNA hetero-duplex is suitable for use in the practice of the invention. An exemplary enzyme suitable for the practice of the invention is RNaseH (also known as, e.g., ribonuclease H and endoribonuclease H); other suitable enzymes include, for example, RNaseI, RNaseIII, Nuclease S1, T7 Exonuclease, and Exonuclease III (Exo III), or any combination there of. RNaseH is a small, 18.5 kD enzyme with robust activity and high specificity that degrades RNA that is hybridized to DNA in a DNA/RNA heteroduplex, and substantially does not degrade single stranded RNA or RNA/RNA duplexes (Goodrich, T. T., Lee, H. J., & Corn, R. M. Direct detection of genomic DNA by enzymatically amplified SPR imaging measurements of RNA microarrays. Journal of the American Chemical Society 126, 4086-4087 (2004)). RNaseH is nearly ubiquitous to life and is found in many organisms. RNaseH comes in 2 predominant forms, type I and type II, either of which can be used in this invention. Information about RNaseH as found in different organisms and variants of RNaseH may be found, for example, by searching gene and protein databases using the accession number PF00075. Some commercial suppliers of RNaseH are Sigma, New England Biolabs, Promega, Fermentas, and Epicentre, just to name a few. The specificity for this enzyme against single stranded RNA has been directly tested and found not to degrade probes used for the disclosed assays unless in the presence of a specific sequence of DNA (see FIGS. 8 and 10). The degradation of RNA molecules by RNaseH produces small nucleotide polymers, usually single ribonucleotides, di-ribonucleotides, and tri-ribonucleotides, which represents nearly full degradation of the original RNA probe. RNaseH is well characterized and commercially available from a number of sources. Thermostable RNaseH is also commercially available from Epicentre and has a higher temperature optimal catalysis than other forms of RNaseH (e.g., higher optimal catalysis than E. coli RNaseH). Embodiments of the methods disclosed herein use the degradation of RNA probes with sequences complementary to specific DNA biomarkers for detection of target nucleic acid sequences.

The methods, compositions, systems and devices disclosed herein find use in the identification and quantization of a target DNA or RNA polynucleotide in a sample, such as in a pool of sequences including one or more target sequences, which may be unrelated polynucleotides. Quantization of specific nucleic acid samples may be achieved by comparing the total signal (fluorescent or otherwise) obtained during the assay with a standard curve of known polynucleotide target concentrations. Specific examples of applications include the detection of pathogenic viruses, bacterial, or other micro-organisms, the detection of polymorphisms in the human or other animal populations, and detection and quantization of other organisms through the detection of their biomolecules such as DNA or RNA which are indicative of the presence of said targets. Assays having features of the invention may be used to detect and identify the presence of specific DNA sequences and may be used in assays for diagnosis of many types of infection and disease. For example, such assays may be used for food quality assurance testing and for the detection of pathogenic bacteria such as salmonella, E. coli, or other harmful bacteria.

Signal amplification may be achieved in the assay, for example, by triggering the degradation of many probes per target polynucleotide molecule in a sample. Essentially, once the first probe has been degraded, it diffuses away from the target sequence, since there is not enough hybridization energy to keep it bound. After diffusion of the degraded probe, another probe will hybridize to the target polynucleotide and for be degraded. In this sense the signal is amplified and the target sequences are catalytic. A probe used in an assay can be varied in sequence and length to accommodate many systems and different polynucleotide targets. In one aspect, if the polynucleotide target comprises DNA, the probe will comprise and RNA domain in the oligonucleotide probe. In another aspect, if the polynucleotide target is RNA, the probe will comprise a DNA domain in the oligonucleotide probe. For example, a DNA probe can be made to screen for RNA sequences by using T7 endonuclease, which cleaves an RNA/DNA hetero-duplex. This however will degrade the target molecule. Furthermore, this invention can be done directly in solution and can be multiplexed with a variety of different colored dyes or other detectable label. Also, if samples need to be cleaned or isolated, a biotin molecule could also be attached to the probe which would allow specific polynucleotide isolation by binding to streptavidin surfaces or beads before RNaseH treatment. These embodiments also have the advantage that unligated probes need not necessarily be removed, as in the absence of the target, no significant amplification will occur. Furthermore, the disclosure provides a simplified method that does not require (although may be included) separation of cleaved probes from uncleaved probes prior to detection of a target polynucleotide.

The methods, compositions, and kits disclosed herein provide advantages over other assays presently available. Due to the simplicity of the assays, the lack of need for DNA amplification, and the clear-cut detection criteria, the assays disclosed herein may be performed by non-specialists. The methods, compositions, and kits disclosed herein provide a molecular assay suitable for the detection of HPV, HIV, chlamydia, gonorrhea, or other nucleic acid targets of interest in clinical samples with high accuracy, rapid turnaround, low cost, and ease of use.

In another aspect of the invention the assays, methods, compositions, and kits disclosed herein use RNA or RNA chimeric probes in conjunction with the enzyme RNaseH and other suitable enzymes to identify polynucleotide targets, such as polynucleotide targets indicative of particular bacteria, viruses, or other organisms. Many variations of the probe architectures are suitable so that cost, stability, and signaling (e.g. optical, fluorescent) features can be considered in the ultimate configuration. The three exemplary methods described utilize the unique property of RNaseH to specifically degrade RNA which is hybridized to DNA in a heteroduplex. It will be recognized that the agent or enzyme used for cleavage of a heteroduplex can be modified based upon the type of target polynucleotide and probe as well as conditions of the assay. Such selection can be performed using the teaching described herein as well as skill generally known in the art.

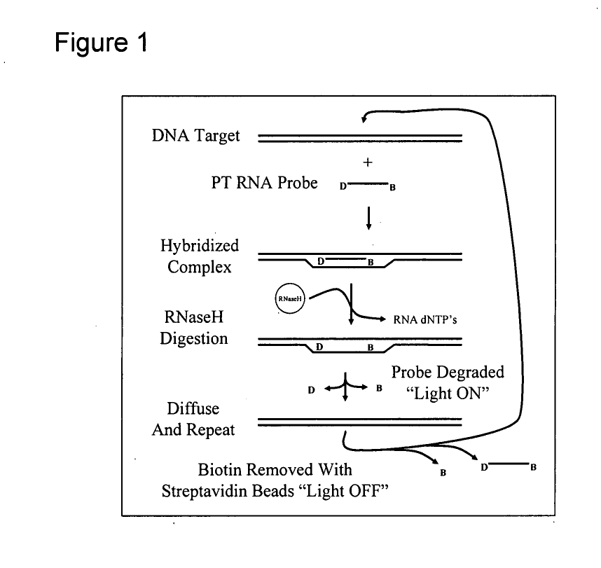

The unique property of RNaseH has been exploited for detection by others (Goodrich, T. T., Lee, H. J., & Corn, R. M. Direct detection of genomic DNA by enzymatically amplified SPR imaging measurements of RNA microarrays. Journal of the American Chemical Society 126, 4086-4087 (2004)). RNaseH does not degrade single stranded RNA. The first technology, Probe Trapping (PT) uses a small RNA molecule with a dye coupled to one end and a biotin molecule on the other (see FIG. 1). The second technology, Hybrid Energy Transfer (HET), uses a small RNA molecule with a dye coupled to one end and a gold nanoparticle on the opposite end. The third method, Fluorescent Probe Degradation (FPD), utilizes a small RNA molecule which has fluorescent base analogs incorporated into the sequence which become highly fluorescent upon RNaseH degradation of the probe. Each of these methods relies on RNaseH degradation of the probe only when in the presence of a complementary DNA sequence which then triggers detectable signal such as fluorescence. Importantly, because the target DNA is not degraded, the disclosed assays provide a basis for significant and direct amplification by the simple addition of more RNA probe.

The first example demonstrating the invention, Probe Trapping (PT), uses a small RNA molecule with a dye coupled to one end and a biotin molecule on the other (see FIG. 1). The second example demonstrating the invention, Hybrid Energy Transfer (HET), uses a small RNA molecule with a dye coupled to one end and a gold nanoparticle on the opposite end. The third example, Fluorescent Probe Degradation (FPD), utilizes a small RNA molecule which has fluorescent base analogs incorporated into the sequence which become highly fluorescent upon RNaseH degradation of the probe. Each of these exemplary methods uses RNaseH degradation of the probe. The probe is degraded when in the presence of a complementary DNA sequence which, upon degradation, triggers a detectable signal (e.g., such as fluorescence). Because the target DNA is not degraded, the assays provide a basis for significant and direct amplification by the simple addition of more RNA probe.

The methods, compositions, systems, and devices of the invention utilize, in one aspect, the unique properties of nucleases, such as RNaseH, to degrade probes only in the presence of a specific nucleic acid target sequence. Probe degradation is then measured and quantitated, correlating to the amounts of target molecules. For example, probes specific to the target sequence with an RNA portion are degraded by RNaseH. The degraded probe is quantitated and correlates to target amounts.

In the past, two solutions have been commonly used to address the need for efficient assays which detect specific DNA sequences: 1) amplification of the target molecule through processes like PCR, and 2) signal amplification through enzymatic processes of colorimetric amplification or using photo-multiplier tubes. While these methods have been successful, many impose a new layer of complexity to the assay which limits it applicability.

The assays disclosed herein are inherently less burdened by the issues mentioned above, since they rely on the catalytic ability of an enzyme or enzymes working on the target substrate. With the technology disclosed herein the target DNA sequence is unmodified by probe hybridization or RNaseH degradation and the assays are catalytic since multiple probes can be degraded by a single template. A degraded probe does not maintain enough hybridization energy to keep it in the heteroduplex and a second probe can then bind to the same target molecule and the process is repeated. Repetition of this cycle can be repeated, leading to large levels of amplification.

Such assays are suitable for use in assays for other infectious diseases or biomarker detection such as HPV, HIV, chlamydia, gonorrhea, hepatitis B, and other diseases, contaminants, and biomarkers. In addition, these assays have uses for non-medical and biomolecular detection as well, including use for biohazard identification and forensics, both of which could use DNA markers as identification of the presence of a specific bioterrorist pathogen or as identification of the presence of DNA evidence in a criminal investigation. These assays are believed to be robust and efficient, and to provide a cheaper and more accurate means of DNA and pathogen detection.

These technologies enable the detection of single-stranded or double-stranded nucleic acid in a sequence-specific manner. This may be accomplished by hybridizing the target nucleic acid molecule to a probe molecule to form a probe-target complex. Such a complex has an RNA/DNA heteroduplex component and occurs only when sequence homology between the probe and the target are sufficient to overcome the entropy from salt, temperature, and other factors in the hybridization conditions. Use of an agent such as a nuclease, ribonuclease, or deoxyribonuclease can be employed to degrade the scissile nucleic acid. The scissile nucleic acid structure is typically a nucleic acid portion of the probe which can be degraded into at least 2 parts. Degradation is typically enzymatic degradation, usually accomplished by the action of a nuclease. Degradation means that the original scissile nucleic acid linkage was at one time a single part and after degradation, the scissile nucleic acid linkage has been divided into at least 2, or more, parts. Degraded subunits are usually mono-nucleotides, di-nucleotides, or tri-nucleotides for RNaseH. The scissile nucleic acid linkage may also separate the ends of the probe and/or may be the linking component for all moieties conjugated onto the probe so that upon degrading the probe into 2 or more fragments, moieties diffuse apart from each other.

An exemplary scheme for assays having features of the invention is presented in FIGS. 1, 2, and 3 showing the generation of fluorescence following enzymatic degradation of an RNA probe. For the PT assay, the biotin allows removal of dye attached to intact probe, but degraded probes release the dye to provide a signal indicating degradation after removal of intact probes with streptavidin beads.

FIG. 1 provides an example of a Probe Trapping Assay having features of the invention. An RNA probe of complementary sequence is added to a sample containing the target DNA, and allowed to hybridize. Addition of RNaseH degrades the probe only when in the DNA/RNA heteroduplex. All biotin is removed from solution with streptavidin beads; this removes all intact probes with dye appendages. If the probe has been degraded due to the presence of the complementary DNA sequence, a large fluorescent signal is observed from the free dye. The DNA template is unchanged and can be used to degrade multiple probes, leading to signal amplification.

As illustrated in FIG. 1, a DNA target is mixed with a probe trapping (PT) RNA probe having dye “D” and biotin “B”. The PT RNA probe hybridizes with the target DNA to form a hybridized complex. Addition of RNaseH allows digestion of the RNA probe that is hybridized to the target DNA. Dye “D” may be detected by fluorescence measurements, which may be of autofluorescense, or, preferably, by stimulated fluorescence upon illumination with light of proper wavelengths. Intact probes may be removed by streptavidin; fluorescence measurements made following removal of intact probe may be used to determine the amount of probe degradation by measuring the dye signal from the dye released by enzymatic degradation of the PT RNA probe. Binding of biotin by streptavidin is suitable to sequester the undegraded probe and to remove undegraded probe from the sample-containing solution, or to move undegraded probe to a portion of the solution or portion of the chamber, vessel, or well in which fluorescence measurements may or may not be made, so that fluorescence from undegraded probe will not significantly interfere with fluorescence measurements from fluorescent molecules released by the degradation of the probe. Such fluorescent molecules released by the degradation of the probe provide a signal that indicates the presence of the target nucleic acid sequence, and which may be used to quantitate the amount of target present in the sample.

Thus, the Probe Trapping (PT) technology disclosed herein may use streptavidin beads to remove non-degraded probes from solution (see FIG. 1); other alternatives for removing intact probes from solution include magnetic beads or other precipitating agents and separation techniques (e.g., dialysis membranes). For example, in addition to or in place of biotin/streptavidin, digoxygenin, digoxygenin, dinitrophenol, or other antigen may be attached to a probe and may be recognized and bound by an antibody to digoxin, digoxygenin, dinitrophenol, or other antigen, for removal of undegraded probes. Other antigen and antibody combinations may also be used in the methods disclosed herein.

Probes for this technique may be made with a 5′ fluorescein molecule and a 3′ biotin molecule, for example, or with a 5′ biotin molecule and a 3′ fluorescein molecule. The binding affinity between biotin and streptavidin (10⁻¹⁵ M) makes the coupling between these two molecules very tight, and forms the basis of numerous basic research and diagnostic applications (Gravitt, P. E., Peyton, C. L., Apple, R. J., & Wheeler, C. M. Genotyping of 27 human papillomavirus types by using L1 consensus PCR products by a single-hybridization, reverse line blot detection method. Journal of Clinical Microbiology 36, 3020-3027 (1998)). Streptavidin beads are easily centrifuged and separated from the remaining solution; thus, the biotin-tagged RNA probes are removed from solution. Since the RNA probes have a dye molecule attached to the 5′ end, all dye which is bound to intact RNA probes will also be removed from solution. However, if the probe is degraded, fluorescent molecules no longer attached to the biotin molecules will not be removed from solution with streptavidin beads. These two configurations, the intact probe versus the degraded one, form a basis for an embodiment using optical/fluorescent detection of dye molecules which correlate to the absence or presence of specific DNA sequences.

In FIG. 1, “D” indicates a fluorescent dye and “B” indicates biotin. Different steps of the assay are indicated from top to bottom in the figure. First, sample with a DNA target is mixed with the RNA probe having dye “D” and having biotin “B”, so that both “D” and “B” are attached to the probe while the RNA probe is intact. The RNA probe has a complementary sequence to the target DNA. Addition of RNaseH degrades the probe only when in the DNA/RNA heteroduplex. All biotin is removed from solution with streptavidin coated beads; this removes all intact probes with dye appendages. If the probe has been degraded due to the presence of the complementary DNA sequence, a large fluorescent signal is observe from the free dye. The DNA template is unchanged and can be used to degrade multiple probes, leading to signal amplification.

The indication “Light ON” indicates that light for exciting the fluorescent dye is provided and emission is measured since free dye molecules are present in solution. The indication “Light OFF” indicates that light for excitation is also provided but there is no or very low emission measured since all dye has been removed from solution with streptavidin beads. A first step is illustrated below the first downward-pointing arrow, showing hybridization of the probe to the DNA target to form a hybridized complex. As indicated in FIG. 1, the target DNA may be double-stranded DNA but can also be single stranded. The second downward arrow, crossed by a ball labeled “RNaseH” schematically indicates addition of enzyme, e.g., RNaseH, to the solution including the hybridized complex, and degradation of the RNA probe by the enzyme (indicated by the legend “Probe Degraded”). As indicated by the third downward arrow, with degradation of the RNA probe, the dye “D” and biotin “B” are released. Finally, the dye “D” and biotin “B” are separated allowing separation of intact and degraded probes.

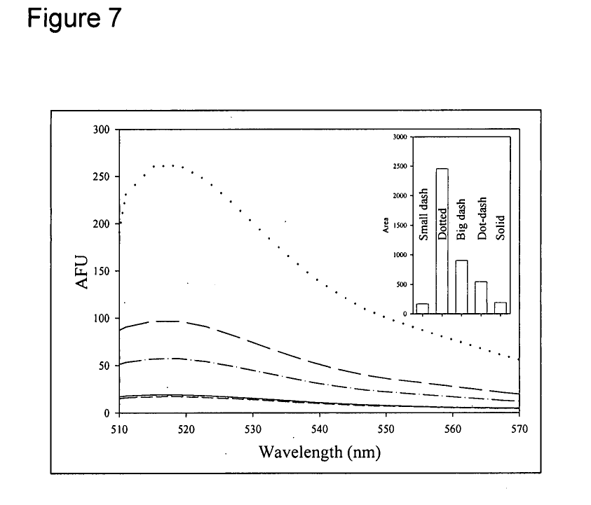

FIG. 7 shows data from a PT assay, illustrating spectra of a PT assay. Fluorescence emission with excitation at 495 nm. 1 pmol PT probe was incubated with varying amounts of DNA and 0.5 units thermostable RNaseH for 2 hours at 61° C. The small dashed line is buffer (0 pM), dotted is 100 nM, big dashed line is 10 nM, dashed-dotted line is 1 nM, and the solid line is 100 pM. Inset shows the area of each plot. Data was repeated, averaged, and summarized in FIG. 8. First, we show that probe can be removed from solution as given by the background scan with just buffer. As DNA targets are introduced to the assay at various concentrations, fluorescent signal accumulates leading to the increase in signal shown in the plot. Data from this experiment is summarized in FIG. 8 and shows signal-to-background calculations. While the signal-to-noise dictates the lower limit of sensitivity for this assay, it could theoretically detect as low as a single molecule of target since the target molecule is unchanged by the assay signal accumulation is depended on incubation times with the enzyme. Thus, very large incubation times can achieve greater sensitivities than what is reported in the figures.

At any stage the biotin can be removed from solution with streptavidin beads followed by centrifugation; any molecule attached to the biotin (remainder of the PT probe) will also be removed from solution. Addition of streptavidin beads allows the streptavidin to bind to the biotin “B”, and allows removal of biotin-streptavidin complexes. Removal of biotin remaining bound to intact probes will also remove dye “D”. Thus, fluorescence measurements of dye “D” after addition of and separation of streptavidin beads provides fluorescence measurements allowing the quantization of the dye “D” that was released due to probe degradation by the enzyme. This correlates to the amount of target molecules in the sample. Hence, all dye is removed from the solution unless the probe has been degraded, so that a fluorescent signal following Streptavidin treatment indicates probe degradation. The intensity of the fluorescent signal can be used to quantitate the amount of probe degradation. As discussed above, RNaseH degradation of the probe requires that the probe be hybridized, so that RNA/DNA hybrids form and can be degraded by the RNaseH enzyme, or other enzyme suitable for use in the assays.

An alternative method for the separation of degraded RNA probes from intact probes uses beads coated with DNA or PNA sequences which are complementary to the PT probe. After RNaseH digestion, the reaction is neutralized or stopped with a divalent metal chelator (e.g. EDTA or EGTA) and samples are incubated with these DNA coated beads which hybridize to all intact probes, and intact probes lacking a biotin. Degraded probes do not hybridize since about 1-3 bases of RNA are typically not sufficient to maintain the required energy to form a duplex. Centrifugation of the beads after incubation with the reaction mixture allows all intact probes to be removed from solution, so that only degraded probe gives signal. This technique has several advantages in the assay including, removal of partial RNA probes which were not purified during synthesis, no biotin/streptavidin protein interactions, and commercial availability. During PT probe synthesis, trace amounts of free fluorescein, free biotin, and probes without biotin are present which may contaminate the assay. Use of DNA or PNA coated beads may aid in the removal of any RNA molecules lacking biotin.

An alternative embodiment of the assays, methods, probes and systems having features of the invention uses a metallic nanoparticle (NP) or quantum dot (QD) to provide or modulate a detectable signal rather than using a dye for the probe trapping (PT) technology. Upon cleavage from the probe, and thus upon isolation from intact probes using methods for the PT assay stated above, the NP is grown or enhanced with metal ions and a reducing agent. In this embodiment, the PT assay uses NP seeds which are grown into large metal particles having a much larger light scattering and optical absorption cross section than initially, enabling them to be detected with suitable optics.

Since there are two stages where specificity is required and the signal is amplified, metallic NP growth version of the PT assay provides many advantages over the others, including potential signal amplification of enormous magnitude (10⁸-10¹⁵), and provides double the specificity and signal enhancement of other methods. The first specificity and signal amplification comes from RNaseH degradation of the probe which liberates many metallic NP per DNA target molecule. The second step for specificity and signal enhancement comes with the metallic growth of NP seeds (seed size of 0.5 nm to 100 nm) and growth up to 0.1-1000 μm.

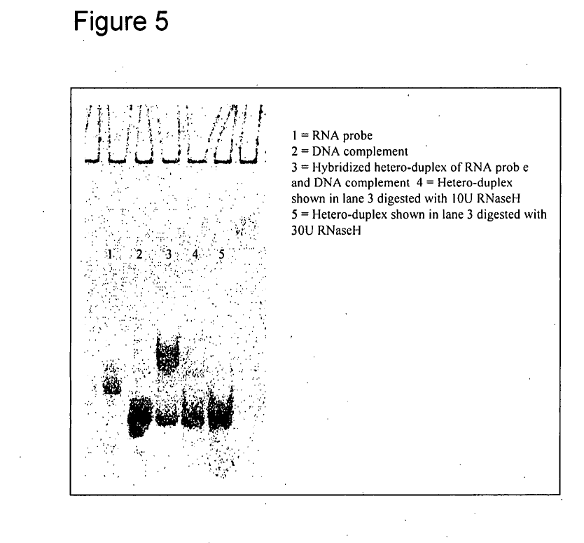

FIG. 5 shows a gel with the lane labeled 1 showing a PT probe; the lane labeled 2 showing the DNA complement; the lane labeled 3 showing the hybridized combination of the PT probe and DNA complement; the lane labeled 4 shows the result of RNaseH digestion of the hybridized PT probe and DNA complement (1 μL RNaseH); the lane labeled 5 shows the result of RNaseH digestion of the hybridized PT probe and DNA complement (3 μL RNaseH).

Further embodiments of the PT assays include the use of an RNA molecule with a dye coupled to one end and a biotin at the opposite end for the detection of specific sequences when RNaseH digests the probe. More specifically, RNA modified at the 5′ and 3′ ends with a biotin and a dye (the “probe”) is used to hybridize to the “target” DNA. RNaseH is added to degrade the probe which only occurs with the RNA/DNA duplex, thereby releasing the dye. Streptavidin beads or surfaces are used to remove intact probes from the sample and only free dye would remain in solution. Detection would be based on fluorescent signal accumulating from free dye in solution only when RNaseH digests the probe, which can only occur if the probe anneals to the correct sequences. The target molecule is not degraded and would also be catalytic. All of the concepts disclosed in the “hybrid energy transfer” disclosure could also be applied to this system.

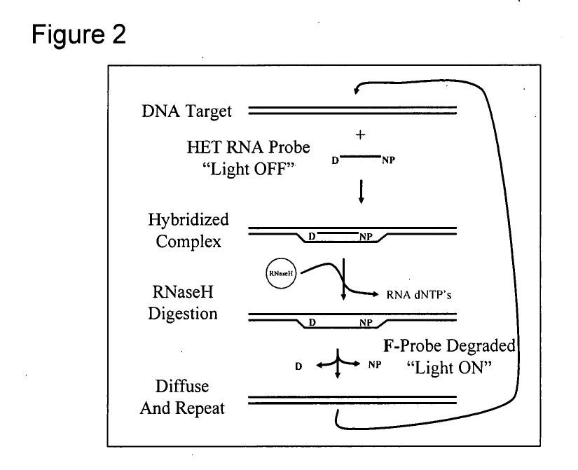

Hybrid Energy Transfer (HET) assays use similar concepts and methods as PT assays but utilize a different probe design. In HET embodiments, the 5′ end of the RNA probe again has a covalently attached fluorescent dye (e.g., fluorescein), but the 3′ end is coupled to a gold nanoparticle (see FIG. 2).

FIG. 2 illustrates an example of Hybrid Energy Transfer. A DNA target of known sequence is added to the RNA probe of complementary sequence and allowed to hybridize. RNA probes are ˜10 nm long and this distance allows efficient FRET between the dye and nanoparticle. RNaseH degradation of the probe causes the dye and gold nanoparticle to diffuse and the distance between the two exceeds FRET capabilities. This leads to a large fluorescent signal from the free dye. The DNA template is unchanged and can be used to degrade multiple probes.

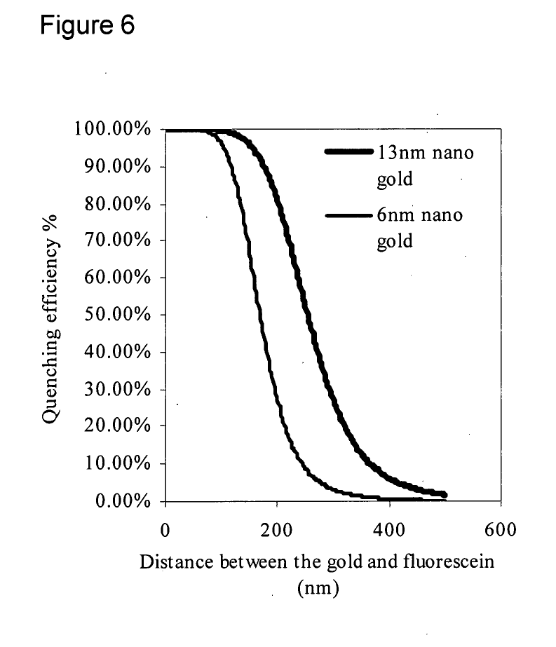

As illustrated in FIG. 2, a DNA target of known sequence is added to a mixture including the RNA HET probe having a complementary sequence. HET RNA probes are about 10 nm long, a distance that allows efficient surface energy transfer (SET) or nanometal surface energy transfer (NSET) (SET and NSET are similar to FRET but operate via a fundamentally different mechanism of energy transfer) between a dye and a gold nanoparticle (about 95%-99% efficient). RNaseH degradation of the probe allows the dye and the gold nanoparticle to separate and to diffuse apart to distances that exceed energy transfer capabilities (e.g., the quenching efficiency, and thus the amount of dye signal that is quenched, is reduced). This leads to a large fluorescent signal from the free dye. The DNA template is unchanged and can be used to degrade multiple probes. As illustrated in FIG. 6, larger gold nanoparticles are able to quench fluorescent signals at greater distances than smaller gold nanoparticles are able to do. FIG. 6 plots quenching efficiency (as %) along the vertical axis, with greater quenching to the top, and separation between a gold nanoparticle and a fluorescein dye molecule on the horizontal axis, with greater separation to the right. The left line illustrates quenching efficiency for 6 nm gold nanoparticles, while the right line illustrates quenching efficiency for 13 nm gold nanoparticles.

An exemplary scheme for nanoparticle assays is presented in FIG. 2. “D” indicates a fluorescent dye such as, for example, fluorescein. “NP” indicates a nanoparticle that is suitable for quenching fluorescence from the dye, such as a metallic (e.g., gold) nanoparticle. A probe is illustrated having both a dye “D” and a nanoparticle “NP”. As illustrated in FIG. 2, a DNA target is mixed with a RNA probe having dye “D” and a nanoparticle “NP”. The RNA probe hybridizes with the target DNA to form a hybridized complex. Addition of RNaseH allows digestion of the RNA probe that is hybridized to the target DNA, allowing separation and diffusion of the dye “D” and nanoparticle “NP”, and reducing or ending the quenching of dye fluorescence by the nanoparticle NP. Dye “D” may be detected by fluorescence. Light ON refers to the correct DNA target being present leading to the generation of a fluorescent signal and Light OFF refers to no DNA target being present and no production of signal or light.

Gold nanoparticles have unique optical properties of light absorption at moderate distances (1-30 nm) through energy transfer (Yun, C. S., Javier, A., Jennings, T., Fisher, M., Hira, S., Peterson, S., Hopkins, B., Reich, N. O., & Strouse, G. F. Nanometal surface energy transfer in optical rulers, breaking the FRET barrier. Journal of the American Chemical Society 127, 3115-3119 (2005). This property has been well documented and can be used to quench the fluorescence of dye molecules such as fluorescein (Ray, P. C., Fortner, A., Gri, J., Kim, C. K., Singh, J. P., & Yu, H. Laser-induced fluorescence quenching of tagged oligonucleotide probes by gold nanoparticles Chemical physics Letters 414, 259-264 (2005) and Dulkeith, E., Ringler, M., Klar, T. A., Feldmann, J., Javier, A. M., & Parak, W. J. Gold nanoparticles quench fluorescence by phase induced radiative rate suppression. Nano Letters 5, 585-589 (2005)). Thus, in an intact HET probe, the dye molecule is heavily quenched due to the proximity of the gold nanoparticle. This quenching effect is much more significant than other quenchers, including the dark quenchers which are commonly used in beacon detection technologies. The background fluorescence is the fluorescence with the HET probe intact, with the gold nanoparticles quenching the dye. Upon probe degradation by RNaseH, the nanoparticle and dye molecules diffuse and the distances between the two no longer allows efficient energy transfer, leading to a large increase in fluorescence from the dye molecule. These two states of the probe are the basis of detection with the intact probe showing a heavily quenched signal and a degraded probe showing a highly fluorescent signal. Thus, the fluorescence signal drastically increases upon degradation of the HET probe by RNaseH which will only occur when a complementary DNA sequence to the probe is present. In further embodiments, the signal can be increased through the use of probe treblers which allow the attachment of multiple dyes to one end of the probe, all of which are quenched by the nanoparticle in the intact probe.

In one aspect, the invention provides a sensor to identify the presence of specific sequences of DNA or RNA by forming hetero-duplexes, in combination with a signaling moiety, usually an organic fluorescent dye or a metallic nanoparticle. One formulation (HET) has a dye at one end of the probe and a nanoparticle at the other end. This state of the probe has a very low fluorescent signal since the energy from the dye is transferred to the nanoparticle and is dispersed without the emission of a photon. However, once the probe (dye/nanoparticle appended RNA molecule) hybridizes to the correct target sequence (DNA molecule to be detected), RNaseH is introduced which specifically degrades the RNA component of the probe causing the nanoparticle and dye to dissociate. This degraded state of the probe has the dye and nanoparticle separated by very large distances leading to very inefficient energy transfer between the dye and the nanoparticle and causing a larger increase in the observed fluorescence signal. The fluorescent dye can be replaced with a semiconducting quantum dot (e.g., CdSe). The difference signal from the dye on the probe between the intact state of the probe and the degraded state of the probe is the basis to determine if a specific DNA sequence is present. The anticipated nucleic acid hybridization segment (target site) is likely to be greater than 15-20 bp, thus making it unlikely that two organic dyes could be used for this sensor in what would be a FRET configuration (where FRET stands for Fluorescence Resonance Energy Transfer, or, equivalently, Förster Resonance Energy Transfer). Energy transfer between dyes and nanoparticles does not follow traditional FRET distance dependence and thus the energy transfer is through a different mechanism, most likely SET or NSET (Yun, C. S., Javier, A., Jennings, T., Fisher, M., Hira, S., Peterson, S., Hopkins, B., Reich, N. O., & Strouse, G. F. Nanometal surface energy transfer in optical rulers, breaking the FRET barrier. Journal of the American Chemical Society 127, 3115-3119 (2005)).

Nanoparticles useful in the practice of the invention include metal (e.g., gold (Au), silver (Ag), copper (Cu), and platinum (Pt), semiconductor (e.g., CdSe, CdS, and CdS or CdSe coated with ZnS) and magnetic (e.g., ferromagnetite, iron (Fe), cobalt (Co), and various alloys and oxide) colloidal materials. Other nanoparticles useful in the practice of the invention include ZnS, PbS, PbSe, ZnTe, CdTe, Cd₃ As₂, InAs, and GaAs. The size of the nanoparticles is preferably from about 2 nm to about 150 nm (approximate diameter) but can be anywhere from 2 nm to 100 um. Shapes other than spheres are included here, such as rods, faceted cubes, and star shapes.

Generation of metal, semiconductor and magnetic nanoparticles are well-known in the art. Gold nanoparticles may, preferably, be synthesized using well known aqueous reduction methods, including citrate-based reduction of a salt in water by heating. (e.g., Mirkin, Anal. Chem., 72, pages 5535-5541 (2000); Schmid, G. (ed.) Clusters and Colloids (VCH, Weinheim, 1994); Massart, R., IEEE Taransactions On Magnetics, 17, 1247 (1981); Hayat, M. A. (ed.) Colloidal Gold: Principles, Methods, and Applications (Academic Press, San Diego, 1991); Ahmadi, T. S. et al., Science, 272, 1924 (1996).

Suitable nanoparticles/nanocrystals are also commercially available from, e.g., Ted Pella, Inc. (gold), Amersham Corporation (gold), Sigma-Aldrich (various), Invitrogen Corp. (various), Evident Technologies (semiconductor), and Nanoprobes, Inc. (gold).

Nanoparticles, the oligonucleotides, or both, may be derivatized to attach the oligonucleotides to the nanoparticles and form the structure for this invention. Such methods are known in the art. For instance, oligonucleotides functionalized with alkanethiols at their 3′-termini or 5′-termini readily attach to gold nanoparticles. Please see Mirkin, Anal. Chem. 2000, 72, pages 5535-5541, Whitesides, Proceedings of the Robert A. Welch Foundation 39th Conference on Chemical Research Nanophase Chemistry, Houston, Tex., pages 109-121 (1995). See also, Mucic et al. Chem. Commun. 555-557 (1996) (a method for binding 3′ thiol DNA to flat gold surfaces for which similarly can be used to bind oligonucleotides to metal nanoparticles). The alkanethiol method (or other linker-thiol modifications, or a variety of coupling chemistries utilizing amines, carboxylic acids, epoxies, aldehydes, etc.; please see Beaucage, Current Medicinal Chemistry, 2001, 8, 1213-1244 for linking chemistries) can be used to bind with metal, semiconductor and metal magnetic colloids listed above. Each nanoparticle will have between one and a plurality of oligonucleotides attached to it (depending on diameter, up to a closely packed monolayer of hundreds on a 15 nm diameter gold nanoparticle, for example). As a result, each nanoparticle-oligonucleotide conjugate can bind to a plurality of oligonucleotides or nucleic acids at the same or sequential time, when having the sufficiently complementary sequence for the use in this invention.

Discussion regarding the synthesis and application of various nanoparticles in the context of nucleic acids is described in Yun, C. S., Javier, A., Jennings, T., Fisher, M., Hira, S., Peterson, S., Hopkins, B., Reich, N, O., & Strouse, G. F., Nanometal surface energy transfer in optical rulers, breaking the FRET barrier. Journal of the American Chemical Society 127, 3115-3119 (2005), and in Braun, G., Inagaki, K., Estabrook, R. A., Wood, D. K., Levy, E., Cleland, A. N., Strouse, G. F., and Reich, N, O., Gold nanoparticle decoration of DNA on silicon. Langmuir 21, 10699-10701 (2005). The same RNA probe sequence as described for the PT results may be used for HET assays as well, with the difference that the RNA probe has a 5′ gold nanoparticle appended. In further embodiments, various nanoparticle sizes (e.g., about 1 nm to about 100 nm in diameter, or about 1.3 nm to about 13 nm) may be used. In further embodiments, various surface modifications of the nanoparticles may also be used, including poly ethylene glycol modifications. Larger nanoparticles are more efficient at the energy transfer process which leads to the quenching of the appended fluorescent dye. The nanoparticles' surface modifications can be important for conferring biostability and biocompatibility (see, e.g., Yin, Y. & Alivisatos, A. P., Colloidal nanocrystal synthesis and the organic-inorganic interface. Nature 437, 664-670 (2005)). The preparation, characterization, and use of nanoparticle-appended nucleic acids has now become routine, as discussed in publications from the inventor's lab (Yun et al., Braun et al. referenced above) and by others (Maxwell, D. J., Taylor, J. R., & Nie, S. M., Self-assembled nanoparticle probes for recognition and detection of biomolecules. Journal of the American Chemical Society 124, 9606-9612 (2002)). Efficient quenching of the fluorescent label by the appended nanoparticles is confirmed optically. Formation of the RNA/DNA heteroduplex may be detected and measured by gel methods (see FIG. 5).

A method for preparing oligonucleotide-nanoparticle conjugate probes is by covalently binding a functional group on the oligonucleotide to the nanoparticle surface. The moieties and functional groups are those which bind by chemisorption or covalent bonding, effecting the attachment, sufficiently permanent, of oligonucleotides to nanoparticles. For instance, oligonucleotides having an alkanethiol or an alkanedisulfide covalently bound to their 5′ or 3′ ends may be used to attach the oligonucleotides to a variety of nanoparticles, including gold nanoparticles. Monolayers or multilayers of reactive molecules, proteins, or polymers on the nanoparticles may provide an alternative attachment layer to the metal surface wherein the oligonucleotides are coupled using chemistry known in the art to create attachment chemical bonds to similarly yield a plurality of oligonucleotides per nanoparticle. Protein coatings such as streptavidin may be used to attach biotinylated oligonucleotides, forming highly stable connections which are non-covalent. Other polymer components may be organic linear, branched, or dendrimeric species such as polyacids, polyamines, or combinations of block copolymers including property modifiers such as polyethylene oxide, and multilayers of polymers, or inorganic polymers such as silica, which particularly may be formed from silanes and/or functional silanes such as aminopropyltrimethoxy silane, and magnetic metal oxides, and copolymers of these. It is advisable however that the thickness of such polymers should be kept minimal to not diminish the distance dependence properties of the energy transfer mechanism. See Mulvaney et al. Chem. Commun., 1996, 6, 731-732 for one method of preparing controlled thickness silica shells over gold nanoparticles, which may be further modified with oligonucleotides on their surface using chemical means available in the art. For example, if an amine functionalized polymer is present it is known that succinimidyl ester chemistry can be used to form a highly stable conjugate in aqueous solution.

To make the conjugates, the oligonucleotides and the nanoparticles are contacted under conditions to allow at least some of the oligonucleotides to bind to the nanoparticles. The invention also includes the nanoparticle-oligonucleotide conjugates produced by this method, methods of using the conjugates to detect and separate nucleic acids, kits comprising the conjugates, methods of nanofabrication using the conjugates, and nanomaterials and nanostructures comprising the conjugates.

The labels may be attached to the probe subunit(s) directly or indirectly by a variety of techniques. The label can be located at the 5′ or 3′ end of the probe subunit, located internally in the probe subunit, or attached to any of one or more spacer arms (of various sizes and compositions) incorporated to facilitate signal interactions. Using commercially available phosphoramidite reagents, one can produce oligomers containing functional groups (e.g., thiols or primary amines) at either the 5′ or the 3′ terminus via an appropriately protected phosphoramidite, and can label them using protocols described in, for example, PCR Protocols: A Guide to Methods and Applications, Innis et al., eds. Academic Press, Ind., 1990. Alternatively, oligomers may have functional groups incorporated off of the nucleotide itself, or may be introduced enzymatically during or post synthesis. These functional groups may be fully functional dyes or reactive groups such as amines or thiols for the subsequent attachment of one or more fluorophores. These methods may also be used to create attachment points to the nanoparticle surface, or a layer to which is bound the nanoparticle.

A wide range of fluorophores may be used in oligonucleotide probes according to this invention. Available fluorophores include coumarin, fluorescein, tetrachlorofluorescein, hexachlorofluorescein, Lucifer yellow, rhodamine, dipyrromethene boron fluoride (BODIPY), tetramethylrhodamine, Cy3, Cy5, Cy7, eosine, Texas red and 6-carboxyl-X-rhodamine (ROX), among other molecules and semiconductor “quantum dots” (commercially available nanocrystals 1-100 nm diameter) undergoing a transitive state susceptible to the proximity effect from the quenching nanoparticles. Fluorophores may be chosen to absorb and emit in the visible spectrum or outside the visible spectrum, such as in the ultraviolet or infrared ranges. More than one type of fluorophore may be incorporated onto the subunits of the oligonucleotide to enhance or tune the excitation and emission characteristics, keeping the spirit of the energy transfer method. There may be a linker, or spacer, between the fluorophore and the attachment point. It is advisable to have this linker be of a size under 50 nm to not diminish the effect of the distance-dependent energy transfer.

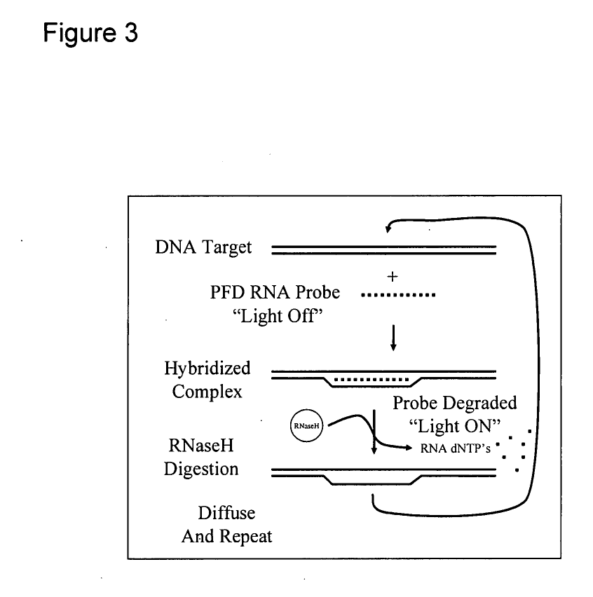

FIG. 3 illustrates an example of Fluorescent Probe Degradation (FPD). A DNA target of known sequence is added to the RNA probe of complementary sequence and allowed to hybridize. RNA probes are composed of nucleotide base analogs which fluoresce such as 2-aminopurine. These base analogs are heavily quenched when in double or single stranded forms. Thus, upon probe degradation the free nucleotides are allowed to fluoresce brightly. The DNA template is unchanged and can be used to degrade multiple probes.

Fluorescent Probe Degradation (FPD) uses fluorescent base analogs incorporated into the RNA probe sequence (see FIG. 3). These base analogs, such as 2-aminopurine (2AP) or pyrrolo cytosine, are well characterized and have been shown to be heavily quenched when stacked into single and double stranded nucleic acids (Allan, B. W. & Reich, N, O., Targeted base stacking disruption by the EcoRI DNA methyltransferase. Biochemistry 35, 14757-14762, (1996)). However, when free in solution as nucleotides, their fluorescence increases ˜10-100 fold (Goodrich, T. T., Lee, H. J., & Corn, R. M., Direct detection of genomic DNA by enzymatically amplified SPR imaging measurements of RNA microarrays. Journal of the American Chemical Society 126, 4086-4087 (2004)). The FPD probe may be synthesized by using T7 RNA polymerase with a 2-aminopurine triphosphate nucleotide.

As illustrated in FIG. 3, a DNA target of known sequence is added to a solution containing a RNA probe having complementary sequence and the probe and target are allowed to hybridize together. FPD probes may include nucleotide base analogs which fluoresce (e.g., 2-aminopurine). These base analogs are heavily quenched when in double- or single-stranded forms. Upon degradation of the probe by RNaseH the free nucleotides are allowed to fluoresce brightly. The DNA template is unchanged by the RNaseH and can be used to degrade multiple probes.

For all assays, PT, HET, and FPD, many variations are possible including different buffer conditions for the assay, different molar rations of probe and DNA target and enzyme, different types of RNaseH, architecture of the probes, and time and temperature of incubation. Normally assays use RNaseH buffers (50 mM Tris-HCl, 75 Mm KCl, and 8 mM MgCl₂, pH 8.2) but could be over a wide range of concentrations from these, usually 10 pM to 1 M for all salts and buffers. pH values are usually around 7-8 but can be done at any pH between pH 3 and pH 11. Temperatures for running the assay are usually somewhere between 37° C. and 50° C. though temperatures between 4° C. and 95° C. could be used and have been investigated. Assays are usually run over 1-2 hours but have given enough signal after ˜5 minutes of incubation and could be done between 5 seconds and 100 hours.

Many steps of the invention require steps of incubation or of sufficient time to allow certain processes to occur. Maintaining the reaction mixture for a sufficient time to allow reaction of the reaction mixture with the target nucleic acid is describing the amount of time needed for probes to hybridize to the DNA target, be digested by the agent or enzyme, and lead to a detectable signal. With DNA targets at very low concentrations, the time for hybridization can be very long, e.g., more than about 30 minutes, or from about one hour, or about two hours, to up to many hours or many days. With high DNA target concentrations, sufficient time may be less than one minute, or about 1 minute, or about 2 minutes, or about three minutes, or about four minutes, or about five minutes, or more, and is usually about 1 to about 5 minutes. In order to discern how much time is needed, a detectable signal needs to be generated. Since the accumulation of signal is time and target concentration dependent, the amount of time needed can be changed to accommodate the system. As stated above, usually 1 hour is standard but can be run from 5 minutes to 100 hours. Sufficient time requires that if the DNA target is present at given concentrations, enough time needs to be given to allow all targets to form the probe-target complexes and have the agent cleave the probes.

Assay variables that are believed to be important to the optimal performance of these assays include the length and sequence of probes, probe to target molar ratios, amount of RNaseH, and incubation times and temperature at various steps (e.g., initial incubation of RNA and sample, exposure to RNaseH). The pH, salt, and buffer conditions for optimal RNaseH activity have been worked out (Gravitt, P. E., Peyton, C. L., Apple, R. J., & Wheeler, C. M. Genotyping of 27 human papillomavirus types by using L1 consensus PCR products by a single-hybridization, reverse line blot detection method. Journal of Clinical Microbiology 36, 3020-3027 (1998)) though they can be altered if needed for a given system.

Many parameters for the exemplary technologies can be modified and adjusted to accommodate the system. While the technologies differ in many ways, some aspects are similar between all assays, PT, HET, and FPD. It will be understood by one of ordinary skill in the art that such conditions may also be varied if desired. Universal properties of the disclosed inventions include chemical bonding between nucleic acid components and signaling or purification components, use of a scissile nucleic acid linker, and probe compositions. Chemical linking between various nucleic acids such as DNA, RNA, LNA, or PNA is standard for one skilled in the art. Most linkages will occur through a phosphor-ester covalent bond between each nucleic acid monomer. Dyes and modifications to nucleic acids will occur through couplings with carbo-phopho-ester bonds or other covalent linkages.

An oligonucleotide according to the methods of the invention may be labeled at the 5′ end or the 3′ end of at least one subunit of the probe. In embodiments, oligonucleotides may be labeled at both the 5′ end and the 3′ end. Alternatively, at least one subunit of the probe may be labeled internally, having at least one, and, in embodiments, more than one, internal label. In embodiments, an oligonucleotide may be labeled at an end and may be labeled internally. The oligonucleotides themselves are synthesized using techniques that are also well known in the art. Methods for preparing oligonucleotides of specific sequence are known in the art, and include, for example, cloning and restriction digest analysis of appropriate sequences and direct chemical synthesis, including, for example, the phosphotriester method described by Narang et al., 1979, Methods in Enzymology, 68:190, the phosphodiester method disclosed by Brown et al., 1979, Methods in Enzymology, 68:109, the diethylphosphoramidate method disclosed in Beaucage et al., 1981, Tetrahedron Letters, 22:1859, and the solid support method disclosed in U.S. Pat. No. 4,458,066, or by other chemical methods using a commercial automated oligonucleotide synthesizer. Modified linkages also may be included, for example phosphorothioates.

Beads used for separation or purification of the DNA target can be of an assortment of sizes and compositions. Bead materials include silica, iron oxide, gold, agarose, polyacrylamide, polymer composites of the type often used for immunoseparation applications, or other solid phase materials commonly used in the field of bead separation. It will be common knowledge to someone trained in the art to find materials to make beads out of. Beads sizes are usually 1 um but can be from 10 nm to 100 um. Beads can be coated with molecules such as partially or fully complementary DNA, RNA, LNA, or PNA to bind the target or probe and enable isolation using separation procedures. Beads can also serve as a support material for gold nanoparticles as a variation of HET. Beads can be functionalized on the surface such as with streptavidin, anti-digoxygenin, or other affinity moieties. In this way, the beads can be used to manipulate or remove assay components. While bead trapping of target molecules for purification purposes is described, beads can also be used to remove intact probes from solution, and thus separating intact from degraded probes. Beads used in all assays shown were from New England Biolabs (product # S1420S) and are 1 micron in diameter and covered in streptavidin. These beads were used to bind to biotinylated probes, incubated for ˜5 minutes to allow hybridization and centrifuged to remove intact probes from solution. Beads could either bind directly to the agent to be removed/manipulated such as a biotinylated probe, or beads can be functionalized with an affinity agent for the target molecule to be removed/manipulated such as a complementary nucleic acid sequence. Thus, beads coated in a complementary sequence to a probe could be used to remove probe from solution since the beads are coated in a molecule which has a high affinity for the probe molecule.

With regard to methods for running the assay, maintaining the reaction mixture for sufficient amount of time is needed for probes to hybridize and become degraded. Sufficient time can be defined as the amount of time needed for a detectable signal to be generated. For this to occur, probes and targets need to hybridize, the cleaving agent must recognize the structure and degrade the probe. Cycling will most likely have to occur with many probes being degraded before sufficient time has occurred, but it is not mandatory. Neutralizing the agent means preventing it from degrading probe even in the presence of the probe-target complex. This can be easily accomplished by heating the sample or using a metal chelator such as ethylene diamine tetraacetic acid (EDTA), ethylene glycol bis(3-aminoethyl ether)-N,N, N′,N′-tetraacetic acid (EGTA) or 1,2-bis(o-aminophenoxy)ethane-N,N,N′,N′-tetraacetic acid (BAPTA) to remove divalent metals required for enzyme catalysis. If other agents are used, other neutralizing strategies will be implemented. Detecting the signal can be accomplished by many methods depending on the label and the composition of the signaling moiety. For a radiolabeled signaling moiety, radiation is measured. For dye signal moieties, intensity and wavelength is measured. Possible chelators include but are not limited to EDTA, EGTA, and BAPTA.

Separation of materials can be accomplished with magnets, dialysis membranes, centrifugation, vacuum, or size exclusion. Separation is defined as compartmentalizing specific reagents so that they do not substantially contaminate adjacent compartments. For example, during separation of intact from degraded probes, separation is desired to reduce the chance of obtaining a false positive, as may occur if small amounts of intact probe are measured with degraded probe. For proper separation, few to no molecules from adjacent compartments are to be measured/scored with desired compartment. Thus, separation of magnetic particles onto the side of chamber where they are not optically measured is sufficient separation since the magnetic particles have been removed from the region which is measured.

For the discussion of probe architectures and compositions, many variations are possible. Critical to all probe architectures are the appendages and chemical moieties which can be attached to the probe (X and Y). These moieties can be appended to either of the ends of the probe (5′ or 3′) or to an internal portion of the probe, such as off the backbone or off of a base internal to the sequence. These moieties are coupled synthetically and will be known by someone skilled in the art. Standard couplings use primary amines or thiol groups in addition to biotin and streptavidin or digoxygenin and anti-digoxygenin. These chemical moieties can be fluorescent dyes such as fluorescein, nanoparticles, quantum dots, affinity agents like biotin, a radiolabel, an enzyme which leads to signal generation, or any other chemical structure which leads to signal production.

NA₁ and NA₂ are the symbols used to represent the nucleic acid portions of the probes which and be composed of any nucleic acid or modified nucleic acid but will usually be made of DNA (deoxyribonucleic acid), RNA (ribonucleic acid), LNA (locked nucleic acid), or PNA (peptide nucleic acid). Compositions can be dispersed of any composition but should not contain nucleic acids which are the scissile nucleic acid linkage unless modified to prevent cleavage. If the scissile nucleic acid linkage is RNA, then NA₁ and NA₂ should not have RNA in them unless modified to prevent cleavage such as a 2′ methyl group. If the scissile nucleic acid linkage is DNA, the NA₁ and NA₂ should not have DNA in them unless modified to prevent cleavage. Nucleic acid potions (NA₁ and NA₂) of the probe can flank the scissile nucleic acid linkage and separate moieties appended to the ends of the probe from the scissile nucleic acid linkage. However, the scissile nucleic acid linkage can be directly next to the attached moieties. These portions of the probe can be almost any length but are usually 5-20 base pairs in length.

In some aspect, the probe has a general architecture: X-NA₁-R-NA₂-Y, or X-NA₁-R, X-NA₁-R-NA₂, or X-R-NA₂. NA₁ and NA₂ are the nucleic acid portions of the probes. The nucleic acid portion of the probe can comprise any nucleic acid base or modified nucleic acid base but will usually be made of DNA (deoxyribonucleic acid), RNA (ribonucleic acid), LNA (locked nucleic acid), or PNA (peptide nucleic acid). Compositions may be dispersed in any suitable composition but preferably do not contain nucleic acids containing the scissile nucleic acid linkage. If the scissile nucleic acid linkage is RNA, then, in embodiments of the invention, NA₁ and NA₂ should not include RNA unless modified to prevent cleavage. If the scissile nucleic acid linkage is DNA, the NA₁ and NA₂, then, in embodiments of the invention, NA₁ and NA₂ should not include RNA unless modified to prevent cleavage, such as with a 2′ methyl group. Nucleic acid potions (NA₁ and NA₂) of the probe can flank the scissile nucleic acid linkage and separate moieties appended to the ends of the probe from the scissile nucleic acid linkage. However, the scissile nucleic acid linkage may be disposed near to, adjacent, or directly next to the attached moieties. These portions of the probe may be of any suitable length, such as, for example, about 5 to about 20 base pairs in length.

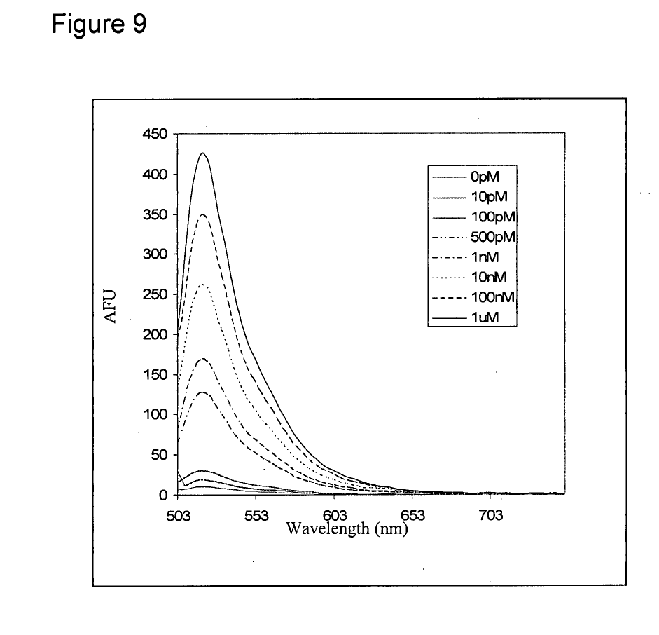

The scissile nucleic acid linkage is a nucleic acid portion of the probe which can be degraded into at least 2 parts. The scissile nucleic acid linkage may be composed of RNA alone, and may be a mixture of RNA, DNA, LNA, or PNA, in any combination, depending on the agent which will degrade the probe. If the agent is a ribonuclease such as RNaseH, the scissile nucleic acid linkage should be RNA unless modified to prevent cleavage such as a 2′ methyl group. If the agent is a deoxynuclease such as Exo III, the scissile nucleic acid linkage should be DNA unless modified to prevent cleavage. Degradation should be enzymatic, usually with a nuclease. Degradation means the original scissile nucleic acid linkage was a single part and after degradation, the scissile nucleic acid linkage is in at least 2 or more parts. Degraded subunits are usually mono-nucleotides, di-nucleotides, or tri-nucleotides for RNaseH. The scissile nucleic acid linkage should also separate the ends of the probe or be the linking component for all moieties conjugated onto the probe so that upon degrading the probe into 2 or more fragments, moieties diffuse apart from each other. For the HET probe and data collected in FIG. 9, RNA was the scissile nucleic acid linkage between the fluorescein and the gold nanoparticle.

Typically, the methods of the disclosure are run under stringency conditions, which allow formation of the first hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, and combinations thereof.

These parameters may also be used to control non-specific binding, as is generally outlined in U.S. Pat. No. 5,681,697. Thus it may be desirable to perform certain steps at higher stringency conditions to reduce non-specific binding.

A variety of hybridization conditions may be used in the disclosure, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tm is the temperature (under defined ionic strength, pH and nucleic acid concentration) at which 50% of the probes complementary to the target hybridize to the polyadenylated mRNA target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of helix destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex such as a psoralen modified DNA molecule.

In one aspect, the target polynucleotide or probe can be immobilized on a solid support (e.g., Corning Microarray Technology (CMT™) GAPS™) or on a microchip. Conditions of hybridization will typically include, for example, high stringency conditions and/or moderate stringency conditions. (See e.g., pages 2.10.1-2.10.16 (see particularly 2.10.8-11) and pages 6.3.1-6 in Current Protocols in Molecular Biology). Factors such as probe length, base composition, percent mismatch between the hybridizing sequences, temperature and ionic strength influence the stability of hybridization. Thus, high or moderate stringency conditions can be determined empirically, and depend in part upon the characteristics of the polynucleotide (DNA, RNA) and the other nucleic acids to be assessed for hybridization. Generally, stringent conditions are selected to be about 5-10° C. lower than the thermal melting point (Tₘ) for the specific sequence at a defined ionic strength and pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts), at pH 7.0 to 8.3 and the temperature is at least about 30° C. for short probes (e.g., 10 to about 50 nucleotides) and at least about 60° C. for long probes (e.g., greater than about 50 nucleotides). Stringent conditions may also be achieved with the addition of destabilizing agents such as formamide. For selective or specific hybridization, a positive signal (e.g., identification of a nucleic acid) is about 2 times background hybridization. For the purpose of this disclosure, moderately stringent hybridization conditions mean that hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe, while the washes are performed at about 50° C. with a wash solution containing 2×SSC and 0.1% sodium dodecyl sulfate. Highly stringent hybridization conditions mean that hybridization is performed at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe, while the washes are performed at about 65° C. with a wash solution containing 0.2×SSC and 0.1% sodium dodecyl sulfate.

The size of the probe may vary, as will be appreciated by those in the art with each portion of the probe and the total length of the probe in general varying from 5 to 500 nucleotides in length. Each portion is between 10 and 100, between 15 and 50 and from 10 to 35 being typically used depending on the use.

Accordingly, the disclosure provides probe set. By “probe set” herein is meant a plurality of hybridization probes that are used in a particular assay. The probe set can be homogenous or heterogeneous.

As will be appreciated by those in the art, nucleic acid analogs find use in probes in the disclosure. In addition, mixtures of naturally occurring nucleic acids and analogs can be made. Alternatively, mixtures of different nucleic acid analogs, and mixtures of naturally occurring nucleic acids and analogs may be made. For example, peptide nucleic acids (PNA) which includes peptide nucleic acid analogs can be used. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched base pairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration.

A hybridization probe may contain any combination of deoxyribo- and ribo-nucleotides, and any combination of bases, including uracil, adenine, thymine, cytosine, guanine, inosine, xathanine hypoxathanine, isocytosine, isoguanine, and the like. In one embodiment, isocytosine and isoguanine are used in primers and probes as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside. For example, components are nearly identical for all assays utilizing sequence-specific probes and are predominantly composed of DNA, RNA, LNA, or PNA. With 5′, 3′, and internal modifications, many options for signaling are possible. Signaling components of probes can be through a radiochemical moiety (examples: ³²P, ³⁵S, ³H), affinity moiety (examples: Biotin, Digoxygenin, Thiol), electrical moiety (example: methylene blue with gold electrode surface), or an optical moiety (examples: Fluorescein, Cy3, Alexa488, Quantum Dots) Optical moieties are usually organic and synthetic fluorophores or quantum dots (QDs). QDs enable long-range, high intensity, multicolor labeling of cellular molecules or probes. MBs provide a powerful and specific synthetic probe strategy with single base-mismatch discrimination.

As will be appreciated by those in the art, target polynucleotides can be obtained from samples including, but not limited to, bodily fluids (e.g., blood, urine, serum, lymph, saliva, anal and vaginal secretions, perspiration and semen) of virtually any organism, with mammalian samples common to the methods of the disclosure and human samples being typical. The sample may comprise individual cells, including primary cells (including bacteria) and cell lines including, but not limited to, tumor cells of all types (particularly melanoma, myeloid leukemia, carcinomas of the lung, breast, ovaries, colon, kidney, prostate, pancreas and testes); cardiomyocytes; endothelial cells; epithelial cells; lymphocytes (T-cell and B cell); mast cells; eosinophils; vascular intimal cells; hepatocytes; leukocytes including mononuclear leukocytes; stem cells such as haemopoetic, neural, skin, lung, kidney, liver and myocyte stem cells; osteoclasts; chondrocytes and other connective tissue cells; keratinocytes; melanocytes; liver cells; kidney cells; and adipocytes. Suitable cells also include known research cells, including, but not limited to, Jurkat T cells, NIH3T3 cells, CHO, Cos, 923, HeLa, SiHa, WI-38, Weri-1, MG-63, and the like (see the ATCC cell line catalog, hereby expressly incorporated by reference).

Methods for introducing oligonucleotide functionalizing reagents to introduce one or more sulfhydryl, amino or hydroxyl moieties into the probe subunit sequence, typically at the 5′ terminus, are described in U.S. Pat. No. 4,914,210. Biotin can be added to the 5′ end by using aminothymidine residue(s), or 6-amino hexyl residue(s), introduced during synthesis, with a suitably reactive (e.g., N-hydroxysuccinimidyl ester of biotin). Labels at the 3′ terminus may employ polynucleotide terminal transferase to add the desired moiety, such as for example, cordycepin ³⁵S-dATP, and biotinylated dUTP.

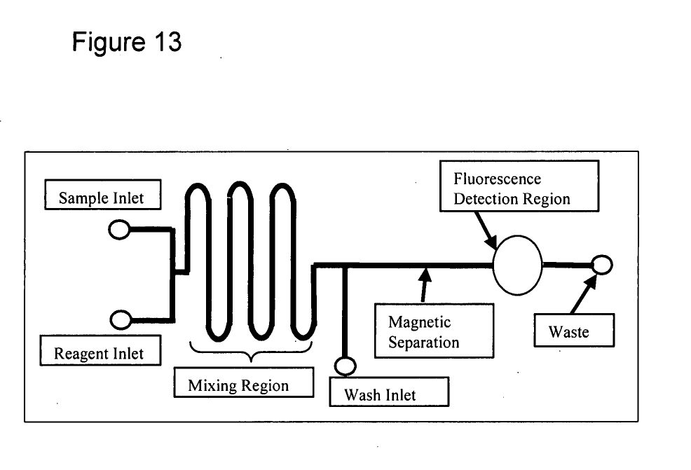

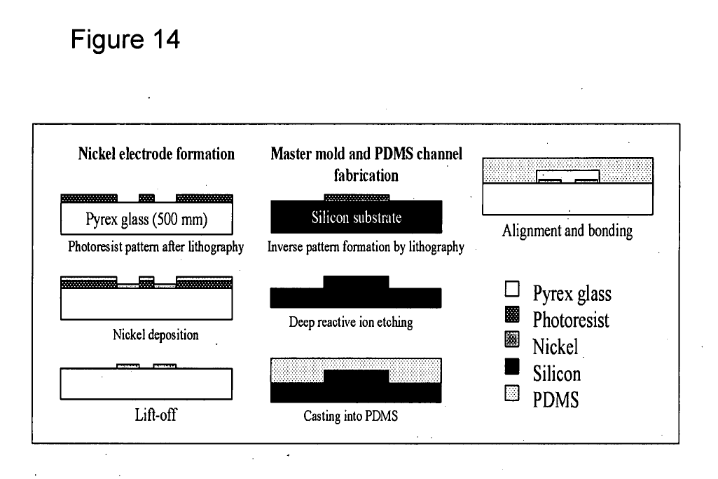

Conducting the assays in a microfluidic device means using very small volumes and chambers. Microfluidic volumes are usually in the 1 μL range but can be from 1000 μL to 1 pL. Schematics of sample microfluidic devices and chambers are provided (see FIGS. 13 and 14). Conducting the assay in a microfluidic device means running all the manipulations (bead removal, addition of enzyme and probe to sample, optical measurements) occur in device which accommodates very small volumes (below 100 μL)

These assays are suitable for use on bacterial cells, cell lysates, and contaminated samples as well. Since many clinical samples are rich with contaminants, it is advantageous that the described assays herein work under these conditions. Although many methods are currently available for DNA extraction and purification from tissues, assays such as those disclosed herein (QIAGEN kits, etc.), which are proficient in analyzing and working with contaminated samples, are very valuable and increases the robustness of the assay. For clinical sample use with the disclosed assays, sample preparation kits may be used. For example, samples suspected of containing pathogenic DNA may be used. Exemplary kits and protocols that can be used include the QIAamp MinElute Virus Spin Kit provided by Qiagen. This kit allows DNA isolation from clinical samples in roughly 1 hour. Other methods for sample preparation are available from suppliers such as Promega.

Polynucleotides may be prepared from samples using known techniques. For example, the sample may be treated to lyse a cell comprising the target polynucleotide, using known lysis buffers, sonication techniques, electroporation, and the like. Many methods for cell lysis are common knowledge for those trained in the art.

A target polynucleotide includes a polymeric form of nucleotides at least 20 bases in length. An isolated polynucleotide is a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an automatically replicating plasmid or virus; or into the genomic DNA of a prokaryote or eukaryote, which exists as a separate molecule (e.g., a cDNA) independent of other sequences, as well as genomic fragments that may be present in solution or on microarray chips. The nucleotides of the disclosure can be ribonucleotides, deoxyribonucleotides, or modified forms of either nucleotide. The term includes single and double stranded forms of DNA.

The term polynucleotide(s) generally refers to any polyribonucleotide or polydeoxyribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide also includes triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

In some aspects a polynucleotide or oligonucleotide (e.g., a probe) includes DNAs or RNAs as described above that contain one or more modified bases. Thus, DNAs or RNAs with backbones modified for stability or for other reasons are nucleic acid molecules. Moreover, DNAs or RNAs comprising unusual bases, such as inosine, or modified bases, such as tritylated bases, to name just two examples, are polynucleotides or oligonucleotides as the term is used herein.

It will be appreciated that a great variety of modifications have been made to DNA and RNA that serve many useful purposes known to those of skill in the art. Polynucleotides and oligonucleotides include such chemically, enzymatically or metabolically modified forms of polynucleotides, as well as the chemical forms of DNA and RNA characteristic of viruses and cells, including simple and complex cells, inter alia.

A target polynucleotide may also be comprised of different target domains that may be adjacent (i.e. contiguous) or separated. The domains can be immediately adjacent, or they may be separated by one or more nucleotides. The terms “first” and “second” are not meant to confer an orientation of the sequences with respect to the 5′-3′ orientation of the target polynucleotide. For example, assuming a 5′-3′ orientation of a target polynucleotide, the first target domain may be located either 5′ to the second domain, or 3′ to the second domain. In addition, as will be appreciated by those in the art, probes on the surface of an array of oligonucleotides or polynucleotides may be attached in either orientation, such that they have a free 3′ end or a free 5′ end. In some embodiments, the probes can be attached at one or more internal positions, or at both ends.

Components of the reaction may be added simultaneously, or sequentially, in any order, with typical embodiments outlined below. In addition, the reaction may include a variety of other reagents which may be included in the assays. Such other reagents include salts, buffers, neutral proteins, e.g. albumin, detergents, and the like, which may be used to facilitate optimal hybridization and detection, and/or reduce non-specific or background interactions. Also reagents that otherwise improve the efficiency of the assay, such as protease inhibitors, nuclease inhibitors, anti-microbial agents, and the like, may be used, depending on the sample preparation methods and purity of the polynucleotides.

In addition, in most embodiments, double stranded target polynucleotides are denatured to render them single stranded so as to permit hybridization of primers and other probes. A typical embodiment utilizes a thermal step, generally by raising the temperature of the reaction to about 95° C., although pH changes and other techniques may also be used. Increases in temperature above room temperature (˜22° C.) from about 25° to 94° C. will increase the population of single stranded forms of a double-stranded target nucleic acid and can be sufficient to enable probe hybridization to the target.

The methods and systems of the invention utilize, in one aspect, the unique properties of nucleases, such as RNaseH, to degrade probes only in the presence of a specific nucleic acid target sequence. Probe degradation is then measured and quantitated, correlating to the amounts of target molecules. For example, probes specific to the target sequence with an RNA portion are degraded by RNaseH. The degraded probe is quantitated and correlates to target amounts.

Since the disclosed assays are quite different from existing assays and rely on a single and robust enzyme (RNaseH, or other suitable enzyme such as a nuclease), the assays disclosed herein are believed to provide advantages and superior performance and reliability as compared to previous assays. A possible source of variability in the disclosed assays may be variability due to contamination from interfering components. For example, introduction of non-specific DNA which could hybridize to the RNA probes and form an RNA/DNA heteroduplex which result in degradation of the RNA probe by RNaseH, leading to a false positive. Also, contaminating RNases other than the exogenously introduced RNaseH could degrade the probe leading to false positives. It is possible to address this possible source of variability by extensive heating of a sample prior to RNA probe addition. Another method for addressing this possible problem is to introduce a decoy, or sacrificial RNA which is not labeled to help prevent non-specific hybridization and to act as a substrate for contaminating enzymes (Gambrai, R. New trends in the development of transcription factor decoy (TFD) pharmacotherapy. CURRENT DRUG TARGETS 5, 419-430 (2004)). A probe decoy may be a DNA molecule which has roughly one third of the identical sequence as the probe on one end. This DNA molecule hybridizes to many of the same non-specific sites that the probe would while still allowing the real probe to out-compete it for the correct site. RNA probe complements may be designed to be shorter than the probe on both ends so that the center section of the probe sequence is hybridized in order to help drive off non-specific binding of the labeled probe by sequestering the center base pairs. In this embodiment of the disclosed assays, the correct target site has sufficient hybridization energy to disrupt the hybridization between the probe and its RNA complement. RNase inhibitors such as the RNasin® Ribonuclease Inhibitor supplied by Promega (Madison Wis. 53711) may also be used to inhibit the contaminating enzymes while not interfering with RNaseH. A further alternative method for sample purification includes providing a trapping probe prior to the use of a detection probe. For example, a digoxygenin-labeled DNA probe complementary to part of the target genome may be used. After cell lysis, such a digoxygenin-labeled DNA probe is added with subsequent use of anti-digoxygenin coated magnetic beads configured for removal from solution with a magnet or centrifugation. Thus, in this way the target DNA molecule may be purified from the cell lysates and any contaminating RNase activity or other contaminants. In further embodiments of the assays of the invention, two or more of these sample preparation methods may be combined in order to remove contaminating RNase activity.

By “substrate” or “solid support” is meant any material that can be modified to contain discrete individual sites appropriate for the attachment or association of oligonucleotides, polynucleotides, or other organic polymers and is amenable to at least one detection method. Possible substrates include, but are not limited to, glass and modified or functionalized glass, plastics (including acrylics, polystyrene and copolymers of styrene and other materials, polypropylene, polyethylene, polybutylene, polyurethanes, Teflon, and the like), polysaccharides, nylon or nitrocellulose, resins, silica or silica-based materials including silicon and modified silicon, carbon, metals, inorganic glasses, optical fiber bundles, and a variety of other polymers. In general, the substrates allow optical detection and do not themselves appreciably interfere with optical detection (e.g., do not fluoresce themselves or quench the fluorescent signal).

Generally the substrate is flat (planar), although as will be appreciated by those in the art, other configurations of substrates may be used as well. For example, three dimensional configurations can be used, for example by embedding beads in a porous block of plastic that allows sample access to the beads and using a confocal microscope for detection. Similarly, the beads may be placed on the inside surface of a tube for flow-through sample analysis to minimize sample volume.

It will be understood that embodiments of the invention include probes having fluorescent dye molecules, fluorescent compounds, or other fluorescent moieties. A dye molecule may fluoresce, or be induced to fluoresce upon excitation by application of suitable excitation energy (e.g., electromagnetic energy of suitable wavelength), and may also absorb electromagnetic energy (“quench”) emitted by another dye molecule or fluorescent moiety. Any suitable fluorescent dye molecule, compound or moiety may be used in the practice of the invention. For example, suitable fluorescent dyes, compounds, and other fluorescent moieties include fluorescein, 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED) and 2′-chloro-7′-phenyl-1,4-dichloro-6-carboxyfluorescein (VIC), cyanine dyes (e.g., Cy³, Cy⁵, Cy⁹, nitrothiazole blue (NTB)), Cys3, FAM™, tetramethyl-6-carboxyrhodamine (TAMRA), tetrapropano-6-carboxyrhodamine (ROX), dipyrromethene boron fluoride (Bodipy), dichloro-fluorescein, dichloro-rhodamine, fluorescein thiosemicarbazide (FTC), sulforhodamine 101 acid chloride (Texas Red), phycoerythrin, rhodamine, carboxytetramethylrhodamine, 4,6-diamidino-2-phenylindole (DAPI), an indopyras dye, pyrenyloxytrisulfonic acid (Cascade Blue), 514 carboxylic acid (Oregon Green), eosin, erythrosin, pyridyloxazole, benzoxadiazole, aminonapthalene, pyrene, maleimide, a coumarin, 4-fluoro-7-nitrobenofurazan (NBD), 4-amino-N-[3-(vinylsulfonyl)-phenyl]naphthalimide-3,6-disulfonate) (Lucifer Yellow), DABCYL, DABSYL, anthraquinone, malachite green, nitrothiazole, and nitroimidazole compounds, propidium iodide, porphyrins, lanthamide cryptates, lanthamide chelates, derivatives and analogs thereof (e.g., 5-carboxy isomers of fluorescein dyes), and other fluorescent dyes and fluorescent molecules and compounds.

Components useful for the operation of devices having features of the invention include: RNA probe construction with a fluorescent dye or a quantum dot and a metallic nanoparticle (such as gold 13 nm NP) coupled so that the dye is heavily quenched (usually <20 nm or 60 b.p. (where “b.p” indicates “base pairs”)). Probe sequences need to be complementary to the DNA target sequences so that hetero-RNA/DNA duplexes can form. RNaseH is introduced to digest the RNA component of the probe. The dye and nanoparticle diffuse into solution where the distance is too great for energy transfer and the dye fluoresces brightly. Since the target DNA molecule is unchanged and can hybridize to another probe after RNaseH digestion, the system can give multiple turnovers of probe degradation per target molecule. A DNA probe could be made to recognize DNA target molecules and T7 endonuclease could be used in the same manner to degrade the probe. See schematic where “D” is a dye and “NP” is a nanoparticle (see FIG. 2).

An exemplary way to broadly practice the invention is to have target samples of DNA or RNA mixed with complementary probes in a microtiter plate, or other sample well. Depending on the nature of the sample, hybridized constructs with a biotinylated probe can be isolated by washing the sample over streptavidin surfaces. Then RNaseH or another nuclease is used to degrade the probe and fluorescence is measured before and after probe degradation by a fluorimeter. Detection can be through a variety of means, ranging from the aforementioned microtiter plate reader, a hand held optical device to excite and detect the fluorescence, or microfluidic devices to carry out similar detection on a much smaller scale.

It will be understood that many variations on the technology disclosed herein are within the embodiments of the invention disclosed herein. While the present disclosure focuses on identifying DNA target sequences, RNA sequences may also be identified by the methods disclosed herein. In addition, one of ordinary skill in the art will recognize that variations can be made in probe design. Aside from the commercially available dyes (in excess of 300), a variety of nanoparticles can be used. Variations of nanoparticle size, composition, and surface group, in combination with variations in dye make for an enormous ensemble of potential probes. A good example of a nanoparticle quenchers are 13 nm gold nanoparticles with poly-ethylene glycol capping group. Also, probe sequence length can be varied from roughly 10 b.p. to over 100 b.p. Probes could have biotin or streptavidin to allow isolation of hybridized complexes. The wide range of commercially available nucleases and RNaseH enzymes (˜500) could also be utilized. The disclosed technology could also be applied to GeneChips technology by attaching a large number of probes of different sequence to a surface in an ensemble of different spots and observing which spots give signal.

The invention disclosed herein includes many novel aspects, including unique combinations of its several components. For example, the use of nanoparticles as energy transfer (ET) partners for dyes is disclosed herein. Current dye based FRET pairs operate in the 10-100 Å regime and two dyes separated by ˜20-30 b.p. have near full emission. In contrast, dye/nanoparticle ET partners in embodiments of the invention typically keep dyes heavily quenched up to 100 Å and partners separated by ˜30-40 b.p. have very low emission. Once the nanoparticle and dye become uncoupled, they diffuse and the distance between these ET partners is concentration dependent and can be made very large where the dye fluoresces almost completely unquenched. Other aspects of the embodiments of the invention include the use of RNaseH to identify specific hetero-duplexes and the nanoparticle's ability to quench over large distances. In one aspect, embodiments of the present invention provide a method for using dye/nanoparticle ET to detect specific nucleic acid sequences.

A trapping agent is defined as a molecule which can be used to separate assay components. An example would be the use streptavidin coated beads and centrifugation to remove intact probes from solution. The trapping agent can be a bead, a surface, or a small molecule and has an affinity for the agent it is trapping. A bead coated in a DNA sequence complementary to the probe could be used since the DNA has an affinity for the RNA probe. Other affinity agents and moieties can be used such as biotin/streptavidin, digoxygenin/anti-digoxygenin, thiols, amines, any of the chemistries described for coupling of probe moieties and dyes, or any other molecule which has a high affinity for the agent to be trapped. Trapping is defined as separation of two components of the assay into separate areas. Trapping agents, once bound/complexed with their target, form a trapping agent-probe complex which implies that physical manipulations to the trapping agent will now affect the agent to be trapped, such as a probe. For example, if the trapping agent is a bead and the agent to be trapped is a probe, after hybridization, removal of the bead from solution will also remove the probe from solution.

In addition, with “hybrid energy transfer” embodiments, for example, an RNA molecule with a dye coupled to one end may be used for the detection of specific sequences when RNaseH digests the probe. More specifically, a RNA modified at one end with a dye (the “probe”) is used to hybridize to the “target” DNA. RNaseH is added to degrade the probe which only occurs with the RNA/DNA duplex, thereby releasing the dye. Using large (˜1 micron) beads coated with DNA sequences complementary to the RNA probe sequence, we are able to separate degraded probe from intact probe. Intact probes maintain enough energy to remain hybridized to the DNA sequences appended to the beads while degraded probes do not. Thus, by centrifugation and removal of the beads, intact RNA probes with the attached dye are also removed from solution. Detection would be based on fluorescent signal accumulating from free dye in solution only when RNaseH digests the probe, which can only occur if the probe anneals to the correct DNA sequence. The target molecule is not degraded and would also be catalytic.

In embodiments of the methods, other components may be included, such as a digoxygenin probe for viral genome purification and other systems for the dye or label. Using a digoxygenin labeled DNA probe which is complementary to the target DNA sequence and anti-digoxygenin coated beads, target DNA molecules are cleaned up and isolated from contaminants that may be present, especially if isolated from a lysed cell. This may enable purification of DNA targets from contaminating RNase activity or other contaminants. This could also be used to concentrate the DNA target if needed. Other suitable variations include variations in the means of signaling in addition to fluorescence. Instead of, or in addition to, using a dye that is appended to the probe, a radioactive label (e.g., ³H, ¹³C, ¹⁸O, ³²P, ¹²⁵I, or other radiation source) may be used as a signal. These radio isotope labeling techniques can be measured directly or further amplified through radio/fluorescence or some other means. Other signaling components could be radicals or gold nanoparticles. Gold nanoparticles can be used as seeds for metallic growth for signal. If degraded probe produced free gold nanoparticles, using silver enhancement or some other metallic growth method, optical detection through absorbance can be achieved. Other variations include different chemistries for attachment of a nanoparticle, a dye, or a biotin. Coupling chemistry can be accomplished through amide-carboxylic acid dehydration (peptide bond formation) or straight thiol chemistry.

Further embodiments of the PT assays include the use of an RNA molecule with a dye coupled to one end and a biotin at the opposite end for the detection of specific sequences when RNaseH digests the probe. More specifically, a RNA modified at the 5′ and 3′ ends with a biotin and a dye (the “probe”) is used to hybridize to the “target” DNA. RNaseH is added to degrade the probe which only occurs with the RNA/DNA duplex, thereby releasing the dye. Streptavidin beads or surfaces are used to remove intact probes from the sample and only free dye would remain in solution. Detection would be based on fluorescent signal accumulating from free dye in solution only when RNaseH digests the probe, which can only occur if the probe anneals to the correct sequences. The target molecule is not degraded and would also be catalytic. All of the concepts disclosed in the “hybrid energy transfer” disclosure could also be applied to this system. Furthermore, the previous disclosure has all the background, state-of the art, and inventor information.

RNaseH enzymes are found in many organisms; any RNaseH may be used in the practice of the invention. RNaseH comes in two predominant forms, type I and type II, either of which can be used in this invention. Information about RNaseH as found in different organisms and variants of RNaseH may be found, for example, by searching gene and protein databases using the accession number PF00075, IPR002156, PS50879, or the enzyme code (EC 3.1.26.4). Other protein folds derived from RNaseH which could be used for the invention can be found using the accession number IPR012337 or GO:0004523. Some commercial suppliers of RNaseH are Sigma, New England Biolabs, Promega, Fermentas, and Epicentre, just to name a few. RNaseH cleaves the 3′-O—P-bond of RNA in a DNA/RNA hetero-duplex. The products are a 3′-hydroxyl and 5′-phosphate terminus. RNaseH is a non-specific endonuclease and catalyzes the digestion of RNA molecules by a hydrolytic mechanism. Unlike other ribonucleases, RNaseH leaves a 3′-phosphorylated nucleic acid. RNaseH is nearly ubiquitous to life and can be found in nearly all organisms including archaea, prokaryota, and eukaryota. In eukaryotic DNA replication, RNaseH is responsible for removal of the Okazaki fragments.

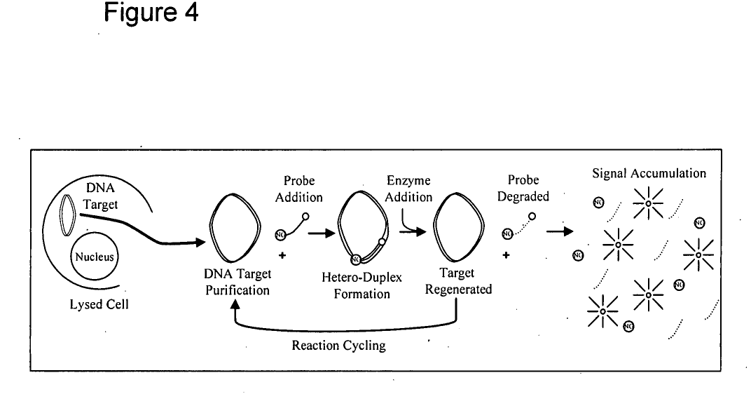

The Probe Trapping (PT) technology may use streptavidin coated beads to remove non-degraded probes from solution (see FIG. 1). Streptavidin coated beads can be removed from solution efficiently by either centrifugation or using magnetic beads (Kalle W. H. J., Hazekampvandokkum, A. M., Lohman P. H. M., Natarajan, A. T., Vanzeeland, A. A., & Mullenders, L. H. F. The Use of Streptavidin-Coated Magnetic Beads and Biotinylated Antibodies to Investigate Induction and Repair of DNA Damage: Analysis of Repair Patches in Specific Sequences of UV-Irradiated Human Fibroblasts. Analytical Biochemistry 208, 228-236 (1993)). The probes for this technique were made with a 5′ fluorescein molecule and a 3′ biotin molecule. Although fluorescein was selected as the dye, many other dyes could be substituted here if unforeseen problems arise. Fluorescein also is highly characterized with robust coupling chemistry to nucleic acids allowing for an economical and robust modification to an RNA probe. For the HET assay, fluorescein also has very nice spectral overlaps with gold nanoparticles, making this dye the best partner for energy transfer with gold nanoparticles (see, e.g., FIGS. 4 and 5). The binding affinity between biotin and streptavidin (K_{d}˜10⁻¹⁵ M) makes the coupling between these two molecules very tight, and forms the basis of numerous basic research and diagnostic applications. Streptavidin coated beads are easily centrifuged and separated from solution and if the biotin-tagged RNA probes are bound, they are also removed from solution. Since the RNA probes have a dye molecule attached to the 5′ end, all dye which is bound to intact RNA probes is also removed from solution upon streptavidin bead centrifugation. However, when the probe is degraded, the fluorescent molecules are no longer attached to the biotin molecules and can no longer be removed from solution with streptavidin coated beads. These two scenarios, the intact probe versus the degraded one, forms the basis for the optical/fluorescent detection of dye molecules which correlate to the absence or presence of specific DNA sequences.

All patents and publications cited herein, both supra and infra, are hereby incorporated by reference herein in their entirety.

### EXAMPLES

Synthesis of nanoparticles for the HET probe used in FIG. 9 was as follows. Approximately 13 nm diameter Au nanoparticles (AuNP) were synthesized through a citrate reduction (C. K. Grabar, R. G. Freeman, M. B. Hommer, and M. J. Natan. Preparation and characterization of Au colloid monolayers, Analytical Chemistry, 67, 735, (1995)) and phosphine capping (G. Schmid, and A. Lehnert, The complexation of gold colloids, Angewandte Chemie-International Edition in English, 28, 780 (1989). Briefly, a 500 mL aqueous solution of 1 mM HAuCl₄ was prepared and brought to reflux under vigorous stirring. Then 50 mL of 38.8 mM trisodium citrate was added. The heat was removed after 15 min with continued stirring. After cooling to room temperature, 150 mg of BSP (bis(p-sulfonatophenyl)phenylphosphine dihydrate, dipotassium salt, Fluka) was added over a period of 5 min followed by overnight stirring. Small amounts of a 2 M NaCl solution were added to precipitate the particles. After centrifuging, the solids were washed with 250 mM NaCl, brought up in 0.3 mM BSP in H₂O (˜100 nM AuNP). Salt precipitation and NaCl solution wash was repeated, then the solids were redispersed at 400 nM AuNP (using extinction coefficient at 522 nm of 2.43×10⁸ M⁻¹ cm⁻¹) in 10 mM sodium phosphate buffer (pH 7.0)

HET probes were synthesized by activating the 5′-thiol on the RNA precursor with 10 mM TCEP (Pierce) in 10 mM sodium phosphate buffer (pH 7.0) for 30 minutes at room temperature. 200-fold molar excess of RNA (200 μM) was mixed with 20 nM gold nanoparticles in 1 ml of buffer for 16 hours at room temperature. To increase RNA density on the gold surface, salt concentration was gradually increased to 0.1 M by NaCl additions. After 48 hours more of incubation, the excess free RNA molecules were removed with four centrifugations at 14,000 rpm for 25 minutes with resuspension in buffer between each spin. Finally, the RNA modified gold nanoparticles were dispersed in phosphate buffer containing 0.1 M NaCl at room temperature.

PT probes, FPD probes, and the RNA portion of the HET probes were all ordered from Dharmacon (Lafayette, Colo.). For the PT data shown in FIG. 8, DNA target concentrations were varied with 1 pmol PT probe and 0.5 units thermostable RNaseH incubated for 2 hours at 61° C. Enzyme buffers used were standard (50 mM Tris-HCl, 75 Mm KCl, and 8 mM MgCl₂, pH 8.2). Optical measurements were made on the NanoDrop3300 fluorimeter. For the HET assay, 10 fmol (1 nM) of HET probe was incubated with varying amounts of synthetic DNA with 0.043 units of E. coli RNaseH for 1 hour at 37° C. This data was replicated, averaged, and summarized in FIG. 10.

We claim:

1. A method for detecting a single-stranded or double-stranded target nucleic acid which comprises:
(a) contacting a sample comprising a target nucleic acid with an oligonucleotide probe preparation to form a reaction mixture under conditions that allow an oligonucleotide probe in the oligonucleotide probe preparation to hybridize to the target nucleic acid to form a probe-target complex, wherein the oligonucleotide probe preparation comprising a plurality of oligonucleotide probes and an agent that selectively cleaves the oligonucleotide probes upon forming a complex with the target nucleic acid, wherein the oligonucleotide probe comprises one or more analog fluorescence bases;
(b) maintaining the reaction mixture for a sufficient amount of time to allow reaction of the reaction mixture with the target nucleic acid; and
(c) detecting fluorescence in the sample, wherein fluorescence is indicative of the presence of the target nucleic acid.

2. The method of claim 1, wherein the analog fluorescence base is selected from the group consisting of 2-aminopurine (AP), pyrrolo-dC (P-dC), 6-Methyl-3-(β-D-2-deoxyribofuranosyl)pyrrolo[2,3-d]pyrimidin-2-one (pyrrolo cytosine), 4-amino-7-oxo pteridine, 4-amino-6-methyl-7-oxo pteridine, 2-amino-4,7-oxo pteridine, 2,4-oxo pteridine, 2-dimethyl amino-7-oxo pteridine, 3-methyl-isoxanthopterin, and any combination thereof.

3. The method of claim 1, wherein the plurality of oligonucleotide probes are the same.

4. The method of claim 1, wherein the plurality of oligonucleotide probes comprises different sequences.

5. The method of claim 1, 3 or 4, wherein an oligonucleotide probe is between 5 and 500 base pairs in length.

6. The method of claim 1, wherein the agent is a ribonuclease.

7. The method of claim 6, wherein the ribonuclease is RNase H.

8. The method of claim 1, wherein the agent is a deoxyribonuclease.

9. The method of claim 8, wherein the deoxyribonuclease is Exo III.

10. The method of claim 1, wherein the method is performed in a microfluidics device.

11. The method of claim 1, wherein the sample comprises a biological sample.

12. The method of claim 11, wherein the biological sample is from a human.

13. The method of claim 11, wherein the biological sample comprises a virus, bacteria, plant, or any combination thereof.

14. The method of claim 12, wherein the biological sample comprises a vaginal sample.

15. The method of claim 14, wherein the target nucleic acid comprises a genome or fragment of the genome from Human Papillomavirus (HPV).

16. The method of claim 15, wherein the oligonucleotide probe comprises a sequence of about 15 to 30 nucleotides and contains the deoxynucleotide sequence CTAAAACGAAAGTA (SEQ ID NO: 1), or the complement thereof TACTTTCGTTTTAG (SEQ ID NO: 2), or a ribonucleotide sequence CUAAAACGAAAGUA (SEQ ID NO: 3) or the complement thereof UACUUUCGUUUUAG (SEQ ID NO: 4), or any mixture of the two wherein one or more bases of the oligonucleotide probe comprise a fluorescent analog base.

17. The method of claim 16, wherein all or some of the bases of the oligonucleotide probe comprise an analog fluorescent base.

18. The method of claim 1 or 16, wherein the oligonucleotide probe comprises a plurality of consecutive RNA bases comprising one or more fluorescent analog bases and one or more DNA, LNA, PNA or non-fluorescent bases.

19. The method of claim 1, further comprising neutralizing the agent prior to or simultaneously with detection of the fluorescence.

20. The method of claim 19, wherein the agent is neutralized by contacting the sample with a divalent metal chelator.

21. The method of claim 20, wherein the divalent metal chelator is EDTA and/or EGTA.

22. The method of claim 19, wherein the agent is neutralized by heat inactivation.

23. The method of claim 1, wherein the method is conducted in a microfluidic device.

24. The method of claim 23, wherein the microfluidic device comprises:
at least one inlet port;
a mixing chamber, fluidly connected to the at least one inlet port;
a measurement chamber, fluidly connected to the mixing chamber.

25. The method of claim 24, wherein at least the measurement chamber comprises a detection means for detecting fluorescence, isotope emissions, luminescence, color, and any combination thereof.

6. A method for detecting a single-stranded or double-stranded target nucleic acid which comprises:
(a) contacting a sample comprising a target nucleic acid with an oligonucleotide probe preparation to form a reaction mixture under conditions that allow an oligonucleotide probe in the oligonucleotide probe preparation to hybridize to the target nucleic acid to form a probe-target complex, wherein the oligonucleotide probe preparation comprising a plurality of oligonucleotide probes and an agent that selectively cleaves the probes upon forming a complex with the target nucleic acid, wherein the oligonucleotide probe generates a detectable signal change upon cleavage with the agent;
(b) maintaining the reaction mixture for a sufficient amount of time to allow reaction of the reaction mixture with the target nucleic acid; and
(c) detecting a detectable signal in or emanating from the sample, wherein the detectable signal is indicative of the presence of the target nucleic acid.

27. The method of claim 26, wherein the plurality of oligonucleotide probes comprises the general structure:
X-NA₁-R-NA₂-Y, or X-13 NA₁-R, X-NA₁-R-NA₂, or X—R-NA₂
wherein NA₁ and NA₂ comprise PEG linkers, DNA, RNA, PNA, LNA nucleotides or a combination thereof having a length of about 3-100 nucleotides in length, wherein R is a scissile nucleic acid linkage or RNA of about 1-100 ribonucleotides in length, wherein either or both of X and Y generate a detectable signal, wherein X and Y when linked by NA₁-R-NA₂ (i) do not generate a detectable signal, or (ii) generate a signal indicative of an uncleaved probe, whereby upon cleavage with the agent at least one intact oligonucleotide fragment or degraded probe fragment is generated, such fragment being, or being treated so as to be, no longer capable of remaining hybridized to the target nucleic acid, wherein either or both of X and Y generate a detectable signal.

28. The method of claim 27, wherein either X or Y is a surface.

29. The method of claim 28, wherein the surface quenches a fluorescent molecule appended to the probe.

30. The method of claim 28 or 29, wherein the surface is gold.

31. The method of claim 27, wherein either X or Y are particles which enable separation of intact probes from degraded probes.

32. The method of claim 27, wherein either X or Y are styrene beads filled with a fluorescent dye.

33. The method of claim 27, wherein either X or Y are appended to the probe with PEG linkers.

34. The method of claim 27, wherein either X or Y are quantum dots.

35. The method of claim 27, wherein X is a nanoparticle and Y is a fluorescent dye or X is a fluorescent dye and Y is a nanoparticle, wherein the nanoparticle quenches the fluorescent dye through energy transfer when the two are in proximity of 0-100 nm.

36. The method of claim 35, wherein the energy transfer is surface energy transfer (SET).

37. The method of claim 36, wherein the nanoparticle is a gold nanoparticle.

38. The method of claim 36 or 37, wherein a nucleic acid portion of the probe is about 10 nm in length.

39. The method of claim 27, wherein X is a first fluorescent moiety and Y is a second fluorescent moiety, wherein the first and second fluorescent moieties undergo energy transfer when in a proximity of 0-50 nm.

40. The method of claim 39, wherein the energy transfer is fluorescent resonance energy transfer (FRET).

41. The method of claim 27, wherein NA₁ and NA₂ independently comprise from 0 to about 100 deoxyribonucleotides, locked deoxyribonucleotides, peptide deoxyribonucleotides, or PEG linkers, and R comprises from about 1 to 100 ribonucleotides.

42. The method of claim 27, wherein the oligonucleotide comprises a repeating unit of between about 2 to 10 repeat units wherein at least one of NA₁ or NA₂ varies within the probe.

43. The method of claim 26, wherein the agent is a ribonucluclease or deoxyribonuclease.

44. The method of claim 43, wherein the ribonuclease is RNase H.

45. The method of claim 43, wherein the deoxyribonuclease is Exo III.

46. The method of claim 27, wherein X and Y on the probe or probe fragment are selected from the group consisting of a dye, radiolabel, quantum dot, nanoparticle and any combination thereof.

47. The method of claim 46, wherein X and/or Y is selected from the group consisting of an Alexa Fluor Dye, Biotin, Digoxygenin, BODIPY Dyes, Cascade Blue Dyes, Coumarin, Fluorescein (FITC/FAM), Haptens, Lissamine Rhodamine B Dyes, NBD, Oregon Green Dyes, Texas Red Dyes, bimane azide, Marina Blue, Pacific Blue, Rhodamine 6G Dyes, Rhodamine Green Dyes, Rhodamine Red Dyes, Tetramethylrhodamine (TMR/TRITC/TAMRA), 6-carboxy-2′,4,4′,5′,7,7′-hexachlorofluorescein (6-HEX), 6-carboxy-4′,5′-dichloro-2′,7′-dimethoxyfluorescein, (6-JOE), 5-carboxy-X-rhodamine (5-ROX), and 6-carboxy-X-rhodamine (6-ROX).

48. The method of claim 46, where the quantum dot is 1-1,000 nm in diameter and comprised of Cd, S, Se, Te, Zn, Pb, Ni, Co, Fe, Mn, or Pt or any combination thereof.

49. The method of claim 46, where the nanoparticle is 1-1,000 nm in diameter and comprised of Au, Ag, Fe, or Co.

50. The method of claim 46, where the radio label is selected from the group of ³²P, ³³P, ³⁵S, ³H, ¹²⁵I, ¹³¹I, ¹⁴C ¹⁰⁹Cd, ⁴⁵Ca, ⁵⁷Co, ²²Na, ⁶³Ni, ⁸⁶Rb, and ⁵¹Cr or any combination thereof.

51. The method of claim 26, wherein the sample comprises a biological sample.

52. The method of claim 51, wherein the biological sample is from a human.

53. The method of claim 51, wherein the biological sample comprises a virus, bacteria, plant, or combination thereof.

54. The method of claim 52, wherein the biological sample comprises a vaginal sample.

55. The method of claim 54, wherein the target nucleic acid comprises a genome or fragment of the genome from Human Papillomavirus (HPV).

56. The method of claim 55, wherein the oligonucleotide probe comprises a sequence of about 15 to 30 nucleotides and contains the deoxynucleotide sequence CTAAAACGAAAGTA (SEQ ID NO: 1) or the complement thereof TACTTTCGTTTTAG (SEQ ID NO: 2), or a ribonucleotide sequence CUAAAACGAAAGUA (SEQ ID NO: 3), or the complement thereof UACUUUCGUUUUAG (SEQ ID NO: 4), or any mixture of the two.

57. The method of claim 56, wherein some or all the bases of the oligonucleotide probe comprise a fluorescent analog base.

58. The method of claim 26 or 56, wherein the oligonucleotide probe comprises a plurality of consecutive RNA bases comprising one or more fluorescent base analogs and one or more DNA, LNA, PNA or non-fluorescent RNA bases.

59. The method of claim 26, further comprising neutralizing the agent prior to or simultaneously with detection of the detectable signal.

60. The method of claim 59, wherein the agent is neutralized by contacting the sample with a divalent metal chelator.

61. The method of claim 60, wherein the divalent metal chelator is EDTA and/or EGTA.

62. The method of claim 26 wherein the agent is neutralized by heat inactivation.

63. The method of claim 26, wherein the method is conducted in a microfluidic device.

64. The method of claim 63, wherein the microfluidic device comprises:
at least one inlet port;
a mixing chamber, fluidly connected to the at least one inlet port;
a measurement chamber, fluidly connected to the mixing chamber.

65. The method of claim 64, wherein at least the measurement chamber comprises a detection means for detecting fluorescence, isotope emissions, luminescence, color, and any combination thereof.

66. A method for detecting a single-stranded or double-stranded target nucleic acid which comprises:
(a) contacting a sample comprising a target nucleic acid with an oligonucleotide probe preparation under conditions that allow an oligonucleotide probe in the oligonucleotide probe preparation to hybridize to the target nucleic acid to form a probe-target complex,
(b) contacting the sample comprising the probe preparation with an agent to form a reaction mixture, wherein the agent selectively cleaves oligonucleotide probes upon forming the probe-target complex;
(c) maintaining the reaction mixture for a sufficient amount of time to allow reaction of the oligonucleotide probes with the target nucleic acid and the agent;
(d) neutralizing the agent;
(e) contacting the reaction mixture with a trapping agent comprising at least one complementary oligonucleotide linked to a substrate, wherein the complementary oligonucleotide is complementary to at least one intact oligonucleotide probe, under conditions wherein at least one complementary oligonucleotide hybridizes to at least one intact oligonucleotide probe to form a trapping agent-probe complex;
(f) separating the trapping agent-probe complex from the reaction mixture; and
(g) detecting a detectable signal in or emanating from the reaction mixture, wherein the presence of a detectable signal is indicative of the presence of the target nucleic acid,
wherein the oligonucleotide probe comprises a detectable moiety.

67. The method of claim 66, wherein at least one complementary oligonucleotide comprises DNA, RNA, LNA, PNA or any combination thereof.

68. The method of claim 66, wherein the trapping agent comprises a solid support, bead, or nanoparticle linked to the complementary oligonucleotide.

69. The method of claim 68, wherein the solid support bead or nanoparticle is linked to the complementary oligonucleotide magnetically.

70. A method of claim 66, 67, 68 or 69, wherein the method of separation is magnetic, size exclusion, or dialysis membranes.

71. The method of claim 66, wherein the plurality of oligonucleotide probes comprises the general structure:
X-NA₁-R-NA₂-Y, or X-NA₁-R, X-NA₁-R-NA₂, or X-R-NA₂
wherein NA₁ and NA₂ comprise PEG linkers, DNA, RNA, PNA, LNA nucleotides or a combination thereof having a length of about 3-100 nucleotides in length, wherein R is a scissile nucleic acid linkage or RNA of about 1-100 ribonucleotides in length, wherein either or both of X and Y generate a detectable signal.

72. The method of claim 71, wherein either X or Y are appended to the probe with PEG linkers.

73. The method of claim 71, wherein X and/or Y are a fluorescent dye.

74. The method of claim 66, wherein a nucleic acid portion of the probe is about 10 nm in length.

75. The method of claim 71, wherein X is a first fluorescent moiety and Y is a second fluorescent moiety.

76. The method of claim 71, wherein NA₁ and NA₂ independently comprise from 0 to about 100 deoxyribonucleotides, locked deoxyribonucleotides, peptide deoxyribonucleotides, or PEG linkers, and R comprises from about 1 to 100 ribonucleotides.

77. The method of claim 66, wherein the agent is a ribonucluclease or deoxyribonuclease.

78. The method of claim 77, wherein the ribonuclease is RNase H.

79. The method of claim 77, wherein the deoxyribonuclease is Exo III.

80. The method of claim 71, wherein X and Y are selected from the group consisting of a dye, radiolabel, quantum dot, nanoparticle and any combination thereof.

81. The method of claim 71, wherein X and/or Y is selected from the group consisting of an Alexa Fluor Dye, Biotin, Digoxygenin, BODIPY Dyes, Cascade Blue Dyes, Coumarin, Fluorescein (FITC/FAM), Haptens, Lissamine Rhodamine B Dyes, NBD, Oregon Green Dyes, Texas Red Dyes, bimane azide, Marina Blue, Pacific Blue, Rhodamine 6G Dyes, Rhodamine Green Dyes, Rhodamine Red Dyes, Tetramethylrhodamine (TMR/TRITC/TAMRA), 6-carboxy-2′,4,4′,5′,7,7′-hexachlorofluorescein (6-HEX), 6-carboxy-4′,5′-dichloro-2′,7′-dimethoxyfluorescein, (6-JOE), 5-carboxy-X-rhodamine (5-ROX), and 6-carboxy-X-rhodamine (6-ROX).

82. The method of claim 80, where the quantum dot is 1-1,000 nm in diameter and comprised of Cd, S, Se, Te, Zn, Pb, Ni, Co, Fe, Mn, or Pt.

83. The method of claim 80, where the nanoparticle is 1-1,000 nm in diameter and comprised of Au, Ag, Fe, or Co.

84. The method of claim 80, where the radio label is selected from the group consisting of ³²P, ³³P, ³⁵S, ³H, ¹²⁵I, ¹³¹I, ¹⁴C ¹⁰⁹Cd, ⁴⁵Ca, ⁵⁷Co, ²²Na, ⁶³Ni, ⁸⁶Rb, and ⁵¹Cr.

85. The method of claim 66, wherein the sample comprises a biological sample.

86. The method of claim 85, wherein the biological sample is from a human.

87. The method of claim 85, wherein the biological sample comprises a virus, bacteria, plant, or combination thereof.

88. The method of claim 86, wherein the biological sample comprises a vaginal sample.

89. The method of claim 88, wherein the target nucleic acid comprises a genome or fragment of the genome from Human Papillomavirus (HPV).

90. The method of claim 89, wherein the oligonucleotide probe comprises a sequence of about 15 to 30 nucleotides and contains the deoxynucleotide sequence CTAAAACGAAAGTA (SEQ ID NO: 1), or the complement thereof TACTTTCGTTTTAG (SEQ ID NO: 2), or a ribonucleotide sequence CUAAAACGAAAGUA (SEQ ID NO: 3), or the complement thereof UACUUUCGUUUUAG (SEQ ID NO: 4), or any mixture of the two.

91. The method of claim 66, wherein the agent is neutralized by contacting the sample with a divalent metal chelator.

92. The method of claim 91, wherein the divalent metal chelator is EDTA and/or EGTA.

93. The method of claim 66, wherein the agent is neutralized by heat inactivation.

94. The method of claim 66, wherein the method is conducted in a microfluidic device.

95. The method of claim 94, wherein the microfluidic device comprises:
at least one inlet port;
a mixing chamber, fluidly connected to the at least one inlet port;
a separation chamber, fluidly connected to the mixing chamber;
a measurement chamber, fluidly connected to the separation chamber.

96. The method of claim 66 or 95, wherein the trapping agent comprises a metallic bead or nanoparticle linked to the complementary oligonucleotide, and wherein the separation chamber comprises magnetic forces for trapping the trapping agent.

97. The method of claim 95, wherein at least the measurement chamber comprises a detection means for detecting fluorescence, isotope emissions, luminescence, color, and any combination thereof.

98. The method of claim 95, wherein a dialysis membrane prevents the trapping agent-probe complex from entering the measurement chamber.

99. The method of claim 66, wherein the trapping agent-probe complex is separated from the reaction mixture with a dialysis membrane.

100. The method of claim 66, wherein the trapping agent-probe complex is separated from the reaction mixture with a magnet.

101. A method for detecting a single-stranded or double-stranded target nucleic acid which comprises:
(a) contacting a sample comprising a target nucleic acid with an oligonucleotide probe preparation under conditions that allow an oligonucleotide probe in the oligonucleotide probe preparation to hybridize to the target nucleic acid to form a probe-target complex,
(b) contacting the sample comprising the probe preparation with an agent to form a reaction mixture, wherein the agent selectively cleaves oligonucleotide probes upon forming the probe-target complex;
(c) maintaining the reaction mixture for a sufficient amount of time to allow reaction of the oligonucleotide probes with the target nucleic acid and the agent;
(d) contacting the reaction mixture with a trapping agent comprising a complementary oligonucleotide that hybridizes to intact oligonucleotide probes to form a trapping agent-probe complex, wherein the trapping-agent probe complex does not react with the agent;
(e) separating the trapping agent-probe complex from the reaction mixture; and
(f) detecting a detectable signal in or emanating from the reaction mixture, wherein the presence of a detectable signal is indicative of the presence of the target nucleic acid,
wherein the oligonucleotide probe comprises a detectable moiety.


SEQUENCE LISTING (4 sequences)

SEQ ID NO: 1 - DNA, 14 bp, Artificial Sequence
  DNA Probe
ctaaaacgaaagta

SEQ ID NO: 2 - DNA, 14 bp, Artificial Sequence
  DNA Probe
tactttcgttttag

SEQ ID NO: 3 - RNA, 14 nt, Artificial Sequence
  RNA Probe
cuaaaacgaaagua

SEQ ID NO: 4 - RNA, 14 nt, Artificial Sequence
  RNA Probe
uacuuucguuuuag